(19) United States
(12) Patent Application Publication
(10) Pub. No.: US 2007/0082353 A1
(43) Pub. Date: Apr. 12, 2007

(54) GENETIC MARKER SELECTION PROGRAM FOR GENETIC DIAGNOSIS, APPARATUS AND SYSTEM FOR EXECUTING THE SAME, AND GENETIC DIAGNOSIS SYSTEM

(76) Inventors: Yoshiko S. Hiraoka, Kawasaki-shi (JP); Kazunori Miyazaki, Yokohama-shi (JP); Satoshi Itoh, Niihari-gun (JP); Michie Hashimoto, Tokyo (JP); Shunji Mishiro, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/533,134

(22) Filed: Sep. 19, 2006

(30) Foreign Application Priority Data
Oct. 7, 2005 (JP) 2005-295333

(51) Int. Cl. C12Q 1/68; G06F 19/00

(52) U.S. Cl. 435/6; 702/20

(57) ABSTRACT

There is provided a marker selection program for selecting a marker for use in genetic diagnosis. In the program, analysis is carried out by using at least two specimen databases which respectively store data of specimens belonging different populations. By carrying out analysis without integrating all specimen data into single population, information on a minority population can be surely reflected to a gene search. Since the characteristics of each population can be reflected, high-accuracy diagnosing functions can be obtained, providing a practical diagnosing system.

### CROSS-REFERENCE TO RELATED APPLICATIONS

This application is based upon and claims the benefit of priority from prior Japanese Patent Application No. 2005-295333, filed Oct. 7, 2005, the entire contents of which are incorporated herein by reference.

### BACKGROUND OF THE INVENTION

1. Field of the Invention

The present invention relates to a program for selecting a marker suitable for use in genetic diagnosis, a device and a system for executing the program, and a genetic diagnosis system that performs genetic diagnosis by using a selected marker.

2. Description of the Related Art

At present, genetic diagnosis is widely used in various fields, for example, personalized medical care, and systems that predict the effectiveness of a treatment method such as medication based on patients' genetic and clinical data have been invented (for example, Jpn. Pat. Appln. KOKAI Publication No. 2004-113661). One of the biggest problems in constructing such a genetic diagnosis system is how to find a genetic marker associated with a diagnosis item. A method generally performed is such that, for example, all genes are compared between patients and normal subjects or between patients for whom the treatment is effective and patients for whom the treatment is ineffective, and genetic polymorphisms having different occurrence frequencies between two populations are found. Furthermore, a method is performed in which not a single genetic polymorphism but a combination of a plural genetic polymorphisms is used as a marker.

The search for the genetic marker is generally performed by integrating patients' genetic and clinical data into a single statistical population. However, it can be assumed that a gene that controls the tendency to get a disease may differ due to ungraspable environmental factors such as the difference in lifestyle, climate, and dietary habits among regions the patients live in. In addition, it can be easily assumed that even if in the patients suffering from the same disease, a gene that plays an important role in treatment may differ from one another among the patients, because their clinical trial conditions are different from each other due to the difference in a treatment method, the presence or absence of complications, or the like.

In such a case, even if there is a gene that can be a candidate under certain conditions, the gene is hardly recognized after integration of data and the gene may be neglected and missed if the number of the patient under the certain condition is small. As a result, in some cases, important information cannot be obtained at all.

The search for the genetic marker to be used in genetic diagnosis is often performed on a population consists of several tens to several hundreds of patients in which clinical trials conducted. However, when genetic diagnosis is practically used based on the results obtained through the genetic marker search and is started to be used in many medical institutions, the actual diagnosing accuracy often turns out to be lower than initially expected. In such a case, there is a need to re-search genetic markers for use in genetic diagnosis or to re-create diagnosing equations for each individual medical institution or each individual complication. As a result, the application range for genetic diagnosis is narrowed or the practical use of genetic diagnosis is hindered. Moreover, if patient's serum is not stored, a blood specimen needs to be additionally taken, putting an additional burden on the patient. If, for some reason, an additional blood specimen cannot be taken, a clinical trial needs to be done again on another patient population, requiring high cost and an enormous amount of time.

### BRIEF SUMMARY OF THE INVENTION

According to an aspect of the present invention, there is provided a genetic marker selection program which causes a computer to work as functional parts for selecting a marker for use in genetic diagnosis, the functional parts comprising:

a genetic polymorphism data storage configured to store in advance known genetic polymorphisms;

a genetic polymorphism combination list storage configured to store in advance a list of genetic polymorphism combinations each composed of at least two genetic polymorphisms included in data on the genetic polymorphisms;

an allele combination list storage configured to store in advance a list of an allele combination regarding the genetic polymorphism combinations listed in the list of genetic polymorphism combination;

a specimen data storage configured to store, for each of at least two populations to each of which a plurality of specimens belong, a genotype of each specimen assigned from the known genetic polymorphisms and a tendency thereof with regard to a diagnosis item;

an association calculation unit configured to store an allele combination list regarding each genetic polymorphism combination listed in the list of genetic polymorphism combination list and determining whether or not allele combinations listed in the list have a correlation with the diagnosis item based on data stored in a specimen database;

an association listing storage configured to store, in an association listing for each population, the genetic polymorphism combinations and the allele combinations thereof determined to have a correlation by the association calculation unit;

a population comparison unit configured to compare between the association listings for the populations and storing, in a second association listing, the genetic polymorphism combinations and the allele combinations thereof, which are present in all of at least two association listings;

a tendency determination unit configured to select, from the second association listing, the genetic polymorphism combinations and the allele combinations thereof having a same tendency against the diagnosis item in at least two populations, and listing the selected combinations in a third association listing as candidate markers; and

an output unit configured to output the candidate markers obtained by the tendency determination unit.

Preferably, the association calculation unit performs the steps of:

reading the allele combination list for each genetic polymorphism combination in the genetic polymorphism combination list,

classifying specimens based on the specimen database, specimens having each allele combination in the list being classified into group A and other specimens being classified into group B,

classifying specimens both in the case for groups A and for B into an effective group and an ineffective group according to the tendency of the diagnosis item,

testing to determine whether or not there is a significant difference in ratio between the effective and ineffective groups in the groups A and B, and

making an judgment that the genetic polymorphisms and alleles determined to have a significant difference by the test have an association with the diagnosis item.

Preferably, the program further comprises causing the computer, after functioning as the tendency determination unit, to function as a candidate selection unit configured to select an optimum candidate marker for genetic diagnosis from the candidate markers listed in the third association listing.

Preferably, the unit configured to select an optimum candidate marker from the third association listing is a unit configured to average correlation coefficients of the populations and to select a genetic polymorphism combination and an allele combination thereof having a maximum average value of the correlation coefficient.

According to another aspect of the present invention, there is provided a diagnosing function creation program which causes a computer to work as functional parts for creating a genetic diagnosing function, the functional parts comprising:

a diagnosing function creation unit configured to create, for each population and for the candidate markers in the third association listing of claim 1 or 2, a diagnosing function Y=aX+t (where a and t are constants) by setting X=xᵢ=−1 for specimen i belonging to the group A and setting X=xⱼ=+1 for specimen j belonging to the group B, or setting X=xᵢ=+1 for the specimen i belonging to the group A and setting X=xⱼ=−1 for the specimen j belonging to the group B, or setting X=xᵢ=α for the specimen i belonging to the group A and setting X=xⱼ=β for the specimen j belonging to the group B (where α and β are any different numbers), and setting yᵢ=1 or yᵢ=0 for each specimen i based on effectiveness of treatment and/or a tendency to get a disease; and

an output unit configured to output the created diagnosing functions.

In order to select an optimum diagnosing function from the created diagnosing functions, the program is preferably further comprises causing the computer, after functioning as the diagnosing function creation unit, to function as:

a calculation unit configured to calculate a contribution ratio K of the diagnosing function described above to each population;

a selection unit configured to select a diagnosing function of a candidate marker having a maximum average value of the contribution ratio K; and

an output unit configured to output the selected diagnosing function.

According to still another aspect of the present invention, there is provided a system for performing genetic diagnosis on target specimens by using a marker selected as described above. The system comprises:

a reading unit configured to read the marker selected as described above;

an input unit configured to input respective gene sequences of the target specimens which are measured in advance;

a determination unit configured to determine whether or not a genetic polymorphism combination and an allele combination thereof, which are the same as the selected marker, are present in the specimens;

a diagnosing unit configured to perform diagnosis on the specimens based on the determination; and

an output unit configured to output results of the diagnosis.

According to yet another aspect of the present invention, there is provided a system for performing genetic diagnosis on target specimens by using diagnosing functions created as described above. The system comprises:

a reading unit configured to read the diagnosing functions created as described above;

an input unit configured to input respective gene sequences of the target specimens which are measured in advance;

an applying unit configured to apply data on the specimens to the diagnosing functions and obtaining an expected rate; and

an output unit configured to output the obtained expected rate.

The invention relating to the above-described program can also be established as inventions of a device and a system each composed of a computer that executes the program, a method comprising steps which are performed on the computer by the program, and a storage medium having stored thereon the program.

According to the present invention, a marker that can be used in all specimen populations having different conditions can be efficiently selected. In addition, a practical diagnosis system with an excellent diagnosing accuracy and high versatility can be provided.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram of a marker selection system;

FIG. 2 is a schematic chart showing genetic polymorphism combinations;

FIGS. 3A and 3B are schematic charts showing allele combinations;

FIGS. 4A and 4B are schematic charts showing specific examples of allele combinations;

FIG. 5 is a flowchart showing procedure of a method of selecting a marker;

FIG. 6 is schematic charts showing association listings;

FIG. 7 is a schematic chart showing a second association listing;

FIG. 8 is a schematic chart showing a third association listing;

FIG. 9 is a schematic diagram of a marker selection system according to another embodiment;

FIG. 10 is a schematic diagram of a diagnosing function creating system;

FIG. 11 is a schematic diagram of a genetic diagnosis system;

FIG. 12 is a chart showing part of a specimen database in Example 1;

FIG. 13 is schematic charts showing allele combination lists in Example 1;

FIG. 14 is a chart showing part of an association listing in Example 1;

FIG. 15 is a chart showing part of a second association listing in Example 1;

FIG. 16 is a chart showing part of a third association listing in Example 1;

FIG. 17 is charts showing diagnosing functions in Example 1;

FIG. 18 is a chart showing part of an association listing in Example 2;

FIG. 19 is a chart showing part of a second association listing in Example 2;

FIG. 20 is a flowchart of narrowing a search in Example 3;

FIG. 21 is a schematic diagram showing an association between an interferon signaling system and a candidate marker gene;

FIG. 22 is a flowchart for Example 4; and

FIG. 23 shows sequences of four types of transcription factors that are predicted to be bound to a sequence of an OPN gene.

### DETAILED DESCRIPTION OF THE INVENTION

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

In the present invention, genetic diagnosis refers to determining of the effectiveness of certain treatment against a disease in a certain specimen or subject based on the gene sequences of the specimen. Here, the treatment includes chemical therapy by medications or the like, physical therapy such as radiotherapy or the like, and other treatment. The genetic diagnosis also includes predictions on the tendency to get a certain disease and on the degree of progression of a disease when affected with the disease.

In the present invention, matters to be diagnosed by genetic diagnosis, such as those described above, are referred to as diagnosis items. A diagnosis item may be selected if needed. Here, the tendency relative to a diagnosis item indicates, when the diagnosis item is the effectiveness of treatment, for example, whether the treatment is effective or ineffective. When the diagnosis item is the tendency for the specimen to get a disease, the tendency indicates whether the specimen is more likely or less likely to get the disease.

A gene sequence for use in genetic diagnosis is referred to as a marker. For a marker, a gene having a genetic polymorphism is suitably used. The genetic polymorphism includes a single nucleotide polymorphism (hereinafter referred to as a “SNP”), a substitution, a deletion, an insertion, and the like. It is preferable to use an SNP.

In genetic diagnosis of the present invention, not a single genetic polymorphism but a combination of genetic polymorphisms is used as a marker for the following reason. Even if a genetic polymorphism is one that cannot be a marker by itself, an association may be found between a combination of genetic polymorphisms and a diagnosis item by combining a plurality of genetic polymorphisms. In the present invention, a combination of at least two arbitrary polymorphisms is used as a marker. In the present specification, for simplicity, a description is made using a combination of two polymorphisms as an example, but a combination of three or more polymorphisms can also be used similarly.

In the present invention, a marker selection is performed based on specimen databases on at least two populations. Here, the populations indicate specimen populations having different conditions such as environmental factors, treatment methods, or races. For example, each group of specimens hospitalized in a plurality of different medical institutions, such as hospitals A and B, may serve as populations. Alternatively, other classifications such as by race, country, or sex can also be made. It should be noted that the population is not limited to a medical institution, and that the number of populations can be any number greater than or equal to two. Specimen databases are created and stored by respective populations.

Here, the specimen data means data including clinical data such as the gene sequences of individual specimens and the histories of diseases of the specimens, more specifically, the effectiveness of treatment and the tendency to get a disease. Here, the gene sequence of a specimen to be stored in the data may be a genome sequence but may be only a gene sequence in a genetic polymorphism which is currently known to be present in humans.

The present invention will now be described in detail below with reference to the accompanying drawings.

(First Embodiment)

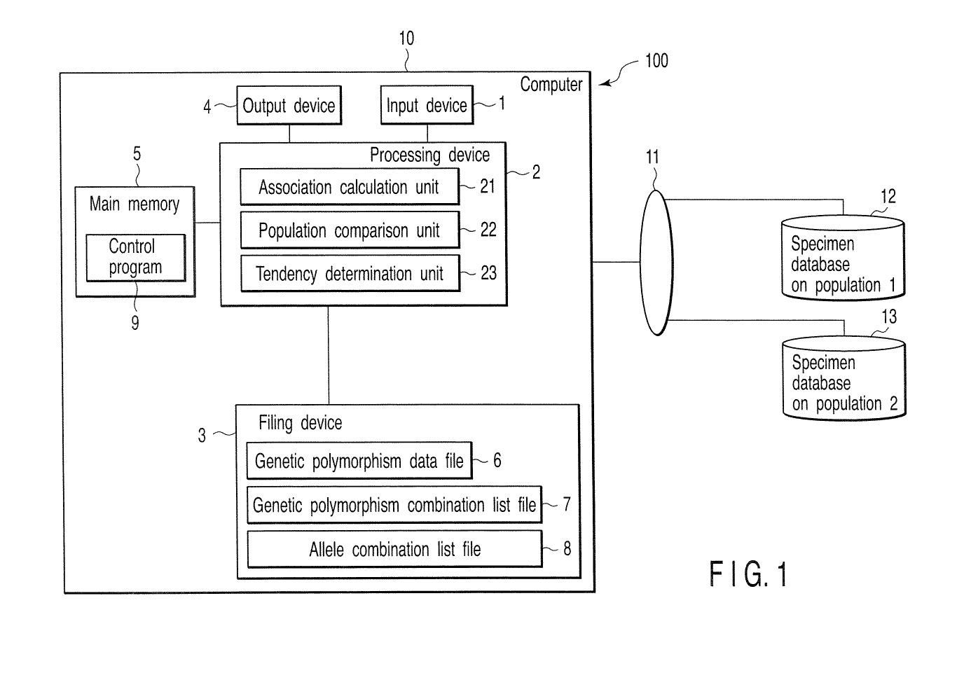

In a first embodiment, there is provided a program for selecting a marker, as well as an apparatus and a system for executing the program. FIG. 1 is a diagram showing an overview of an apparatus and a system for executing a marker selection program of the present invention.

As shown in FIG. 1, a marker selection system 100 includes a computer 10, and a specimen database 12 on population 1 and a specimen database 13 on population 2 which are connected to the computer 10 through a communication network 11.

The computer 10 comprises a processing device 2, and a main memory 5, an input device 1, an output device 4, and a filing device 3 which are connected to the processing device 2.

The computer 10 is implemented by a personal computer, for example. The computer 10 can perform data transmission and reception over the communication network 11 through a communication interface (not shown).

The processing device 2 is implemented by hardware, such as a CPU, that realizes general computer arithmetic operations. The processing device 2 has association calculation unit 21, population comparison unit 22, and tendency determination unit 23.

The main memory 5 has a marker selection program 9 which is stored on an arbitrary storage medium. The computer 10 is controlled by the program 9.

The input device 1 is used to input various data or instructions necessary for processing in the processing device 2. The input device 1 is implemented by, for example, a keyboard, a mouse, and the like. The output device 4 is used to output results or diagnosing results processed in the processing device 2. The output device 4 is implemented by, for example, a display, a printer, and the like.

The filing device 3 has a genetic polymorphism data file 6, a genetic polymorphism combination list file 7, and an allele combination list file 8.

The genetic polymorphism data file 6 has stored therein identification information of genetic polymorphisms which are known to be present in the human genome. The identification information refers to information about a location in a gene sequence where a certain polymorphism is present, the type of base the polymorphism can take, and the like. In the present specification, the identification information is referred to as genetic polymorphism data.

Based on genetic polymorphisms stored in the genetic polymorphism data, all combinations each composed of at least two genetic polymorphisms are created. The created combinations are all listed in a genetic polymorphism combination list. The genetic polymorphism combination list is stored in the genetic polymorphism combination list file 7.

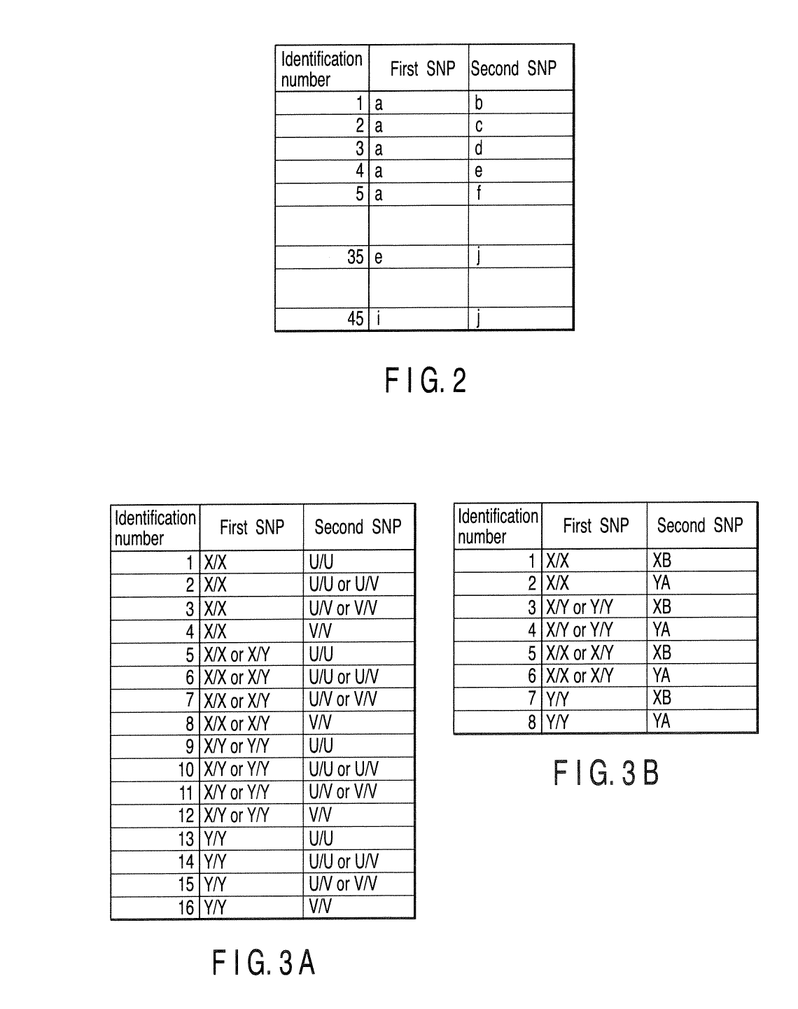

As an example, a combination composed of two SNPs will be described. For example, it is assumed that 10 SNPs: a, b, c, . . . , j are stored in the genetic polymorphism data file. A genetic polymorphism combination list created by using these 10 SNPs is shown in FIG. 2. As shown in FIG. 2, (10×9)/2=45 SNP combinations are created from 10 SNPs. In the genetic polymorphism combination list file 7, a genetic polymorphism combination list such as the one shown in FIG. 2 is stored.

Note that although the above example describes combinations each composed of two genetic polymorphisms, combinations each composed of three or more genetic polymorphisms can also be similarly created.

Now, an allele of a genetic polymorphism is considered. For example, when a first SNP can take a base X or Y, there are three types of alleles, X/X, X/Y, and Y/Y. When a second SNP can take a base U or V, there are three types of alleles, U/U, U/V, and V/V. Therefore, these SNP combinations can take 16 types of allele combinations, as shown in FIG. 3A.

The first combination in FIG. 3 indicates the case where the first SNP is X/X and the second SNP is U/U. The fifth combination indicates the case where the first SNP is X/X or X/Y and the second SNP is U/U.

There is a case where one of the SNPs has two types of alleles, YA or XB. In this case, SNP combinations can take eight types of allele combinations, as shown in FIG. 3B.

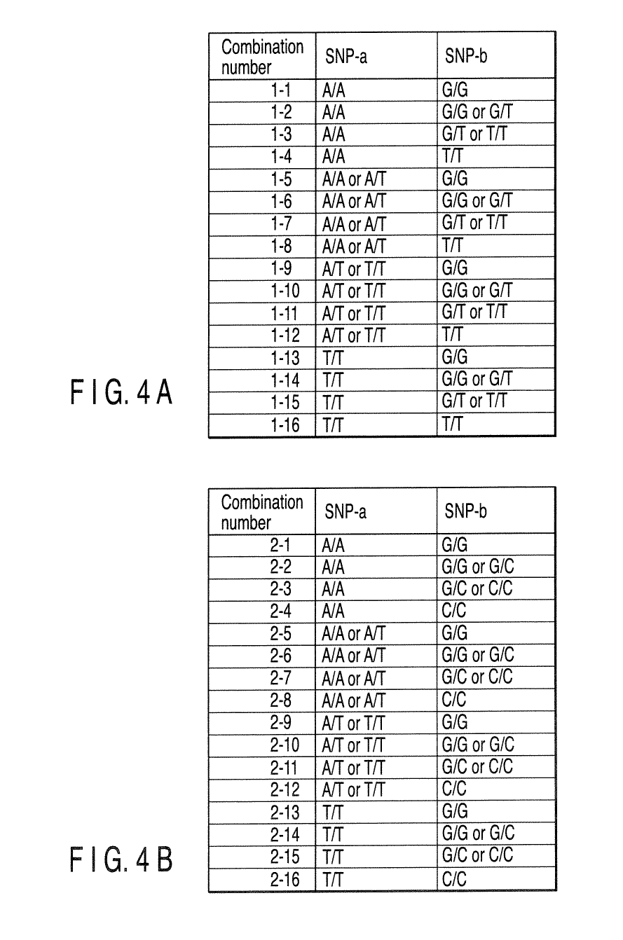

An allele combination list such as those shown in FIGS. 3A and 3B is created for each genetic polymorphism combination. Specific examples of an allele combination list are shown in FIGS. 4A and 4B. FIG. 4A shows a specific example of an allele combination list for the first combination in the genetic polymorphism combination list of FIG. 2. Here, the first SNP takes A and T, and the second SNP takes G and T. FIG. 4B shows a specific example of an allele combination list for the second combination in FIG. 2. Here, the first SNP takes A and T, and the second SNP takes G and C.

When all genetic polymorphisms are single base substitution SNPs, 16 types of allele combinations shown in FIG. 3A are present for each of 45 types of genetic polymorphism combinations shown in FIG. 2. Hence, 45×16=720 types of allele combinations are created in total.

The allele combination lists thus created are stored in the allele combination list file 8.

As shown in the lists of FIGS. 4A and 4B, it is preferred that each allele combination is identified by a combination number. The combination number is created from an identification number of a genetic polymorphism combination and an identification number of an allele combination. For example, the combination numbers can be represented by (1-1) to (45-16). Note that the representation of the combination number is not limited thereto, and any representation can be used as long as the representation can define a genetic polymorphism combination and an allele combination.

The allele combination lists described above are created for all genetic polymorphism combinations and stored in the allele combination list file 8.

Note that these genetic polymorphism combination list and allele combination lists may be created by the processing device 2, or data files which are created in advance may be inputted externally and stored. It is preferable to update the lists when a new genetic polymorphism is found.

Now, the specimen databases 12 and 13 will be described. The specimen databases 12 and 13 are databases created for populations 1 and 2, respectively. In the specimen databases 12 and 13 is stored clinical data such as the gene sequences of individual specimens, the effectiveness of treatment, and the tendency to get a certain disease. The databases are implemented by a magnetic disk, an optical disk, or the like.

The specimen databases 12 and 13 may be stored inside the computer 10; however, it is preferred that the specimen databases 12 and 13 be stored on separate computers or the like owned by the respective populations. In this case, the computer 10 is connected to the specimen databases 12 and 13 through the communication network 11, and necessary data can be obtained.

In view of protecting personal information, it is preferred that data which can be obtained by the computer 10 from a specimen database is limited to only necessary and predetermined data.

The configuration of the computer and system described above is not limited thereto. The configuration can be appropriately changed or modified as long as the configuration allows the program of the present invention to be executed.

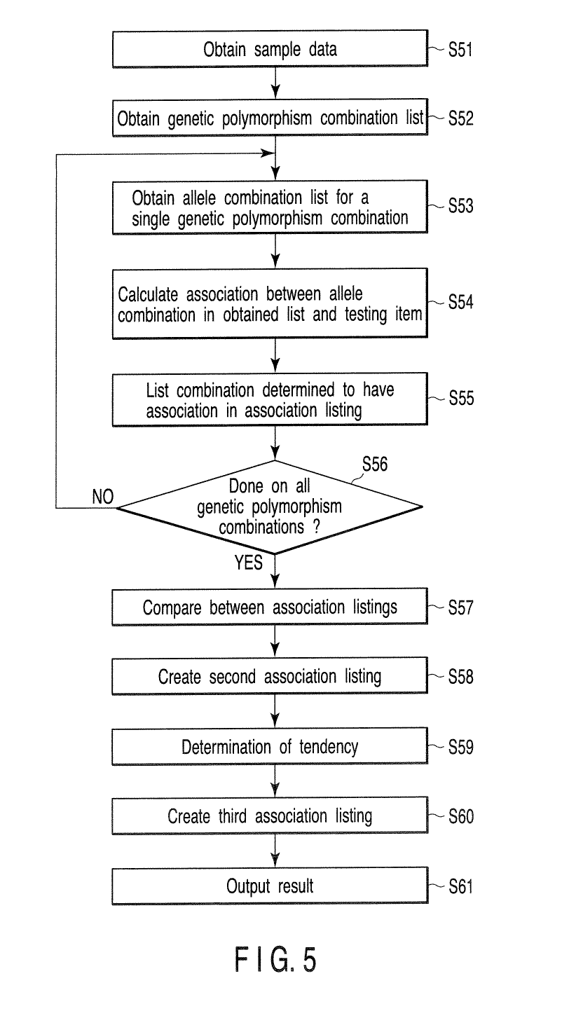

Now, a method of selecting a diagnostic genetic marker using the system of FIG. 1 will be described with reference to a flowchart of FIG. 5. As an example, a method of selecting a genetic marker for predicting the effectiveness of treatment against a certain disease will be described.

First, specimen data is obtained from specimen databases on populations 1 and 2 (S51). A genetic polymorphism combination list is obtained (S52). Note that these steps may be performed in opposite order.

Then, for an arbitrary genetic polymorphism combination in the obtained list, a corresponding allele combination list is obtained (S53). Subsequently, a calculation is performed on all allele combinations in the obtained list to determine whether or not there is a significant association with a diagnosis item (S54). An allele combination that is determined to have a significant association is listed in an association listing by the above-described combination number such that a genetic polymorphism to which the allele combination belongs can be identified (S55). These steps S53 to S55 are performed on all genetic polymorphism combinations (S56).

A specific method of the calculation in step S54 will be described. First, a single combination in the allele combination list is read. Then, the specimen databases are searched, and specimens are classified into a specimen group (group A) having the combination and into other specimen group (group B). The specimens in each group are further classified into a responsive group (SR) in which the treatment is effective and into a non-responsive group (NR) in which the treatment is ineffective, and the number of specimens belonging to each group is counted.

Then, a test is performed to determine whether or not there is a significant difference in efficacy ratio, i.e., the ratio of the responsive group to the non-responsive group, between the groups A and B. The test can be performed using any method. Generally, a chi-square test between two groups is used.

For example, it is assumed that the number of specimens in population 1 is 100 cases which include 45 SR(A) cases, 15 NR(A) cases, 20 SR(B) cases, and 20 NR(B) cases. In this case, the efficacy ratio of group A is 75% and the efficacy ratio of group B is 50%, and therefore, it is determined that group A has higher effectiveness. The result of a chi-square test for this case is P=0.010. Here, given that the case where P<0.05 is determined to be significant, it is determined that the allele combination has a significant association with the diagnosis item.

Alternatively, other methods may be used for the test. For example, the effectiveness Res of treatment is represented by 0 or 1. Specifically, a specimen for whom the treatment is ineffective is set such that Res=0 and a specimen for whom the treatment is effective is set such that Res=1. A genetic polymorphism combination factor S is numerically represented by 1 or 0. Specifically, the specimens classified into group A are represented such that S=1, and the specimens classified into group B are represented such that S=0. In this manner, the correlation coefficient between Res and S and a reliability index of value P are calculated. When the calculation is applied to the above-described case, the correlation coefficient is 0.257 and the value P is 0.010.

A determination as to whether or not there is a significant association can be made by using the value P or the absolute value of a correlation coefficient. For example, when P<0.05, it can be determined that there is a significant association. When the absolute value of a correlation coefficient is 0.3 or greater, it can be determined that there is a significant association. Note that these numeric values that serve as determination references can be appropriately set.

Steps S51 to S56 described above are performed by the association calculation unit 21 of FIG. 1. The calculation and the creation of an association listing are performed individually on each population. Note that the method for the test is not limited to those described above and any other method can be used. The steps through which an association listing is created are not limited to those described in the above example, and may be performed appropriately.

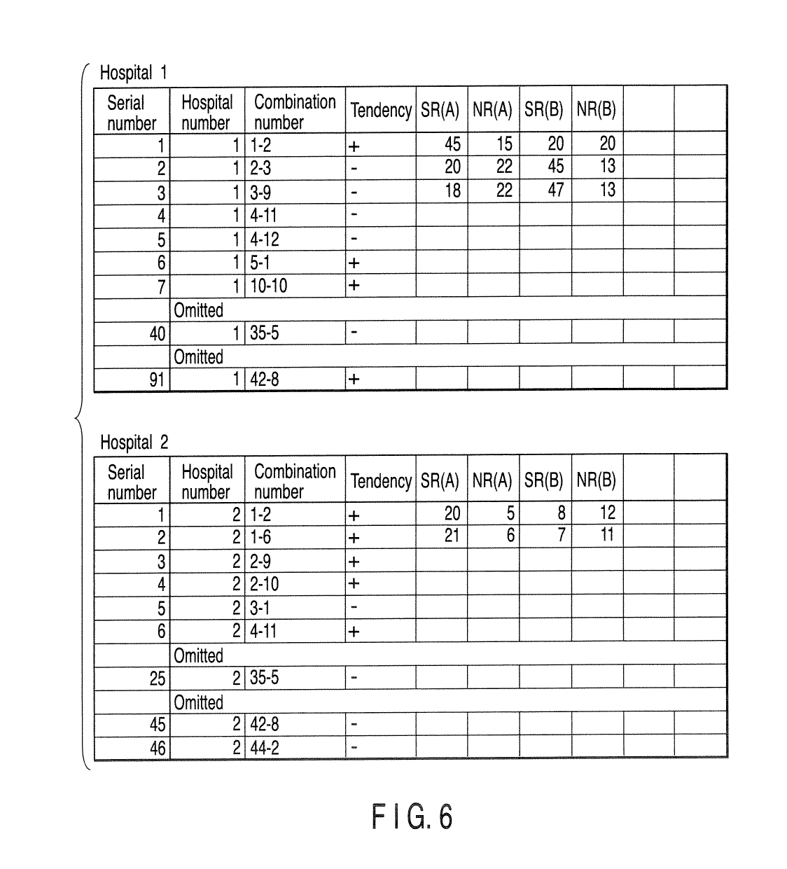

It is preferred that the association listing created in step S55 include a combination number, a tendency of an association with a diagnosis item, a correlation coefficient, or the like as data in a single line. It is desirable to store at the same time information including an index, such as the value P that determines an association, the number of specimens, or the like. FIG. 6 shows specific examples of the association listing. The association listing includes serial numbers for identification, combination numbers, tendency of an association, and the number of specimens for each of response and non-responsive groups in groups A and B.

Here, the tendency of an association is represented as follows. If, as shown in FIG. 6, an independent test between two groups (a chi-square test) is used, the case where the effectiveness of group A is higher than that of group B is represented by “+” and the case where the effectiveness of group A is lower than that of group B is represented by “−”. The case where it is determined that there is no association may be represented by X. More specifically, in combination number 1-2 of hospital 1 of FIG. 6, SR(A)=45 and NR(A)=15. Thus, the effectiveness of group A is 75% and the effectiveness of group B is 50%, and accordingly group A has higher effectiveness. For this case, symbol “+” is entered. If the association listing is created by representing the effectiveness Res of a treatment method by 0 or 1, obtaining a correlation coefficient, and performing a test, the value of the correlation coefficient is, in many cases, entered instead of the tendency. In this case, however, solely symbols for the correlation coefficient may be extracted and entered in the “tendency” column.

Then, by the population comparison unit 22, a comparison is made between the association listing for population 1 and the association listing for population 2 (S57). When there is the same combination number (i.e., a genetic polymorphism combination and an allele combination thereof) in all of the association listings, data for one line about the combination is copied to a second association listing (S58). At this point, there is a need to add to data selected from population 1 a description indicating that the data is derived from population 1, and there is a need to add to data selected from population 2 a description indicating that the data is derived from population 2.

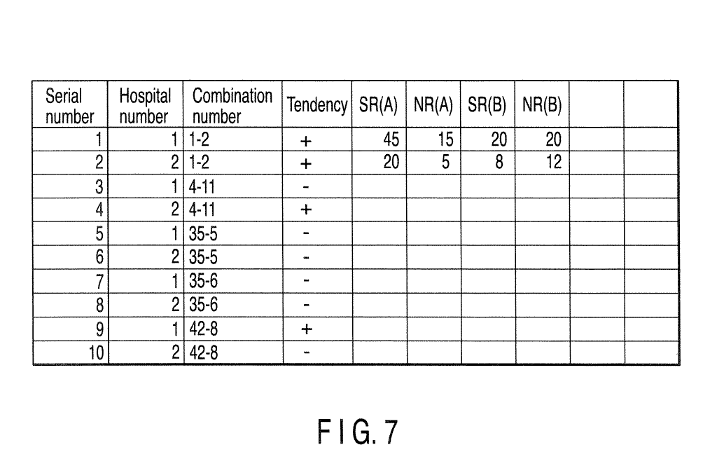

A specific example of the second association listing is shown in FIG. 7. Here, in the “tendency” column, “+” is entered for the case where group A has a higher tendency toward the effectiveness of medications, and “−” is entered for the case where group A has a lower tendency toward the effectiveness of medications. In the example of FIG. 7, combinations that are found in both populations are sequentially listed, but not limited thereto, and the combinations can be stored in any order. However, the order shown in FIG. 7 is convenient in subsequent steps.

Then, by using the tendency determination unit 23, a determination is made as to whether or not each combination listed in the second association listing has the same tendency in all the populations (S59). Specifically, when, in the second association listing, the same symbol be entered in the “tendency” column of all the populations, it is determined that the tendency of the combination toward the effectiveness of treatment is the same. If the test is performed by defining the effectiveness of treatment by 0 or 1 and obtaining a correlation coefficient, it is determined that the tendency is the same in the case where the symbol for the correlation coefficient is the same.

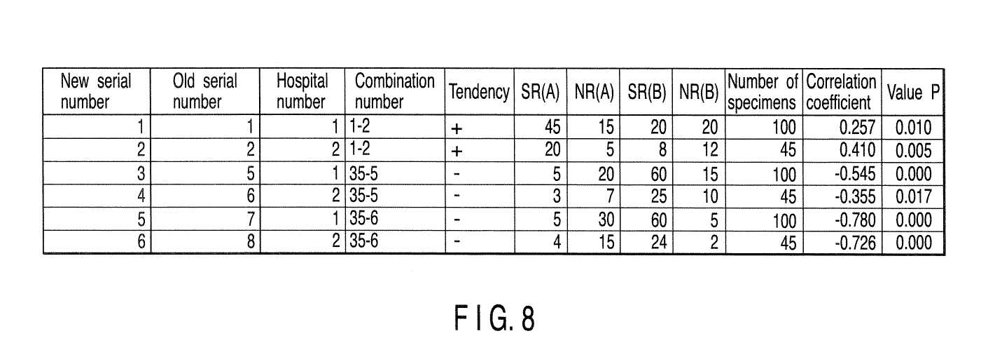

Subsequently, combination numbers that are determined to have the same tendency in step S59 are listed in a third association listing (S60). An exemplary third association listing is shown in FIG. 8. Genetic polymorphism combinations and allele combinations thereof which are represented by the combination numbers listed in the third association listing are candidate markers that can be used as markers for genetic diagnosis. This is outputted by the output device 4 connected to the processing device 2 (S61).

The above-described association listings, second association listing, and third association listing may be stored in the memory of the processing device, or alternatively, dedicated storage may be provided to store the listings.

When there are pluralities of candidate markers listed in the third association listing, all of the candidate markers may be assigned appropriate weights and used. Alternatively, one or two optimum candidate markers may be selected and used. In the latter case, the selection of candidate markers is made based on criteria suitable for objective genetic diagnosis. For example, a combination in which the average of the absolute value of correlation coefficients obtained in populations 1 and 2 is the maximum can be selected. According to this criterion, a combination number (35-6) is an optimum candidate marker in FIG. 8.

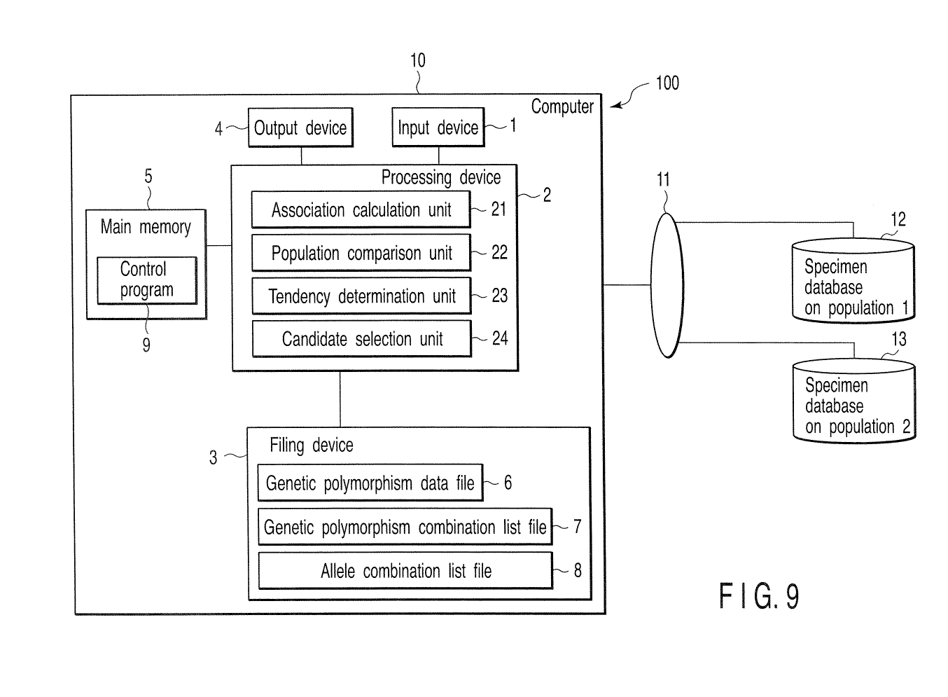

FIG. 9 is a schematic diagram of a marker selection system 100 that has a candidate selection unit 24 for selecting an optimum candidate marker from the third association listing. The candidate selection unit 24 is provided in the processing device 2.

In the marker selection system 100 of FIG. 9, only an optimum candidate marker which is selected by the candidate selection unit 24 may be outputted by the output device 4. Alternatively, the suitability of a candidate marker may be appended to the third association listing, and the listing may be outputted.

In a second embodiment, a program for creating genetic diagnosing functions and an apparatus and a system for executing the program are provided.

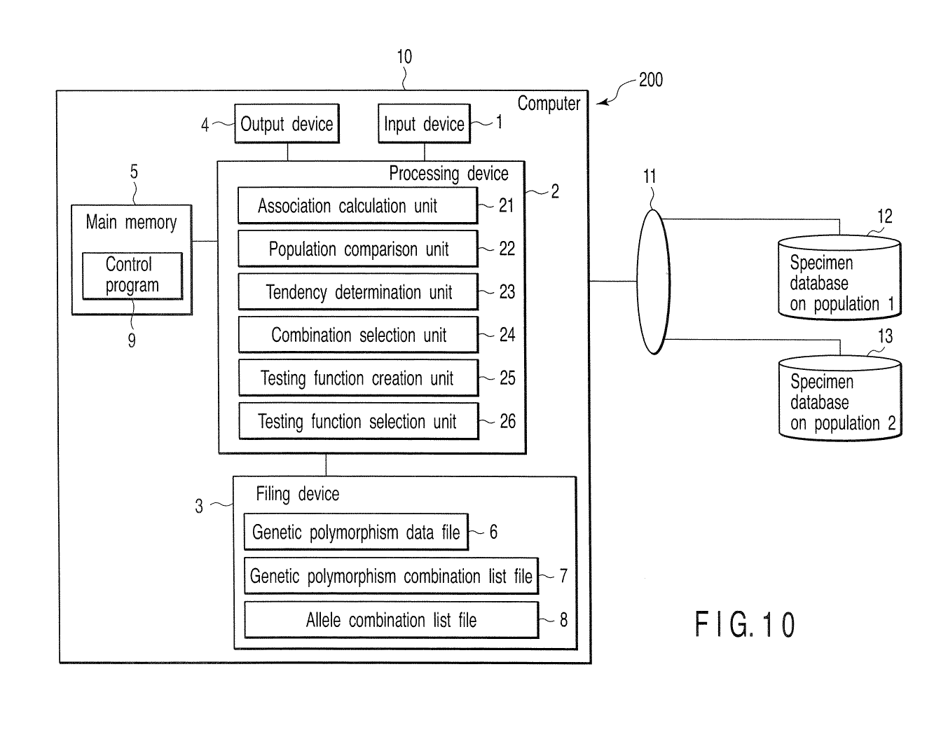

FIG. 10 is a schematic diagram of a diagnosing function creation system 200 for executing a program for creating genetic diagnosing functions. The diagnosing function creation system 200 includes a computer 10, and a specimen database 12 on population 1 and a specimen database 13 on population 2 which are connected to the computer 10 through a communication network 11.

The computer 10 comprises a processing device 2, as well as a main memory 5, an input device 1, an output device 4, and a filing device 3 which are connected to the processing device 2. In the present embodiment, the processing device 2 further includes diagnosing function creation unit 25 in addition to association calculation unit 21, population comparison unit 22, and tendency determination unit 23. In the present embodiment, the configuration of the computer 10 may be the same as that of the above-descried first embodiment, except for the processing device 2.

In the second embodiment, a third association listing is created in the same manner as in the first embodiment. Subsequently, using candidate markers listed in the third association listing, diagnosing functions are created by the diagnosing function creation unit 25. The diagnosing functions are created individually for each population.

Specifically, each candidate marker in the third association listing is classified as follows. A specimen i belonging to the aforementioned group A is set such that X=xᵢ=−1 and a specimen j belonging to the aforementioned group B is set such that X=xⱼ=+1, and for the tendency relative to a diagnosis item, each specimen i is set such that yᵢ=1 or yᵢ=0. For example, an effective group is set such that y=1, and an ineffective group is set such that y=0.

By statistically obtaining a linear regression curve in this manner, a diagnosing function for each population, Y=aX+t (where a and t are constants), is calculated. By this diagnosing function, an expected probability for a diagnosis item is calculated. For example, the probability that the treatment is effective for a certain specimen is calculated.

The diagnosing function may be created for all the candidate markers listed in the third association listing. The created diagnosing functions may be all outputted; furthermore, the most suitable diagnosing function for diagnosis can also be selected.

The selection of a diagnosing function can be made by further providing diagnosing function selection unit 26 in the processing device of FIG. 10. A method of selecting an optimum diagnosing function which is performed by the diagnosing function selection unit 26 will be described below.

First, a contribution ratio K of a diagnosing function created by the diagnosing function creation unit 25 is calculated. The contribution ratio K is a parameter that evaluates the accuracy of the diagnosing function of interest, and can be expressed by the following function:

$$K ≡ \frac{{S}_{yy} - {S}_{e}}{{S}_{yy}}$$

$$where$$

$${S}_{e} = {\sum }_{i = 1}^{n} {\{ {y}_{i} - Y ( {x}_{i} ) \}}^{2}$$

is the residual sum of squares, and]

$${S}_{yy} = {\sum }_{i = 1}^{n} {( {y}_{i} - \overline{y} )}^{2}$$

is the total sum of squares.

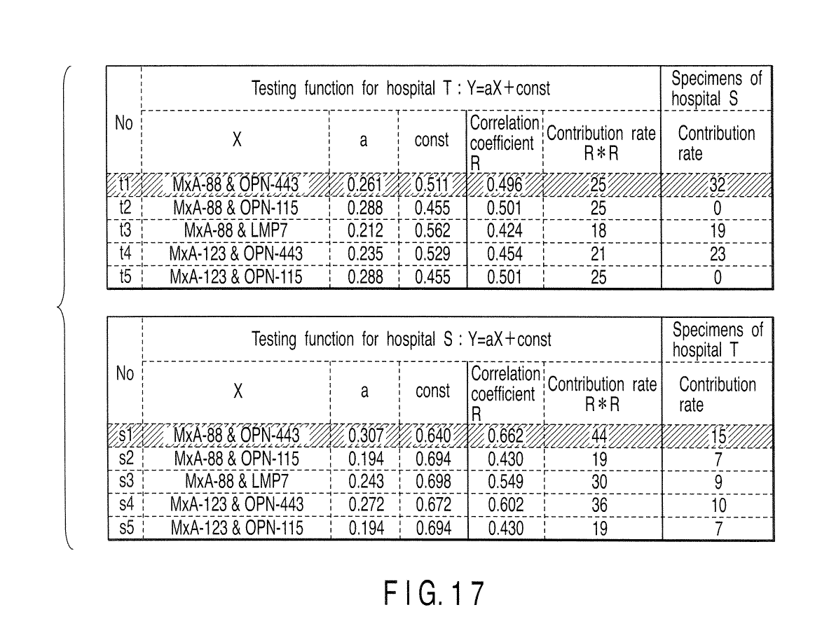

As described above, a diagnosing function is created for each population. Each diagnosing function has a contribution ratio K for each population. For example, a description will be made as follows using a candidate marker of the aforementioned combination number as an example (35-6). The candidate marker has a diagnosing function Y₁ for population 1 and a diagnosing function Y₂ for population 2. Here, the diagnosing function Y₁ for population 1 has a contribution ratio K1₁ for population 1 and a contribution ratio K1₂ for population 2. Likewise, the diagnosing function Y₂ for population 2 has a contribution ratio K2₁ for population 1 and a contribution ratio K2₂ for population 2. Thus, it is understood that, for a single candidate marker, the number of contribution ratios K is the square of the number of populations (see Example 1 and FIG. 17).

Since the contribution ratio K represents the accuracy of a diagnosing function for each population, it can be said that a candidate marker having the maximum average value of four contribution ratios, K1₁, K1₂, K2₁, and K2₂, is the best marker.

Hence, the diagnosing function selection unit 26 calculates the average value of four (when there are three or more populations, the square of the number of populations) contribution ratios K for each diagnosing function, and then selects a candidate marker having the maximum average value as a marker.

Next, a description will be made as to which one of the two diagnosing functions, Y₁ and Y₂, should be used. It is desirable to make a comparison between the average of the contribution ratio K1₁ for population 1 and the contribution ratio K1₂ for population 2 in the diagnosing function Y₁, and the average of the contribution ratio K2₁ for population 1 and the contribution ratio K2₂ for population 2 in the diagnosing function Y₂, and use a diagnosing function of a higher average contribution ratio. Note that when the difference in average value between the two diagnosing functions is not so much, either diagnosing function can be used. The principle is that the same diagnosing function should be used for populations 1 and 2. However, when, for example, K1₁ is significantly greater than K2₁ and K2₂ is significantly greater than K1₂, the function Y₁ can be used for population 1 and the function Y₂ can be used for population 2.

Note that although in the present embodiment a diagnosing function is created for all of the candidate markers in the third association listing, it is also possible to select an optimum candidate marker according to another more suitable selection criteria and then create a diagnosing function for only the selected candidate marker.

In a third embodiment, a program for performing genetic diagnosis on a new specimen subject by using a marker or a genetic diagnosing function described above, as well as a device and a system for executing the program are provided.

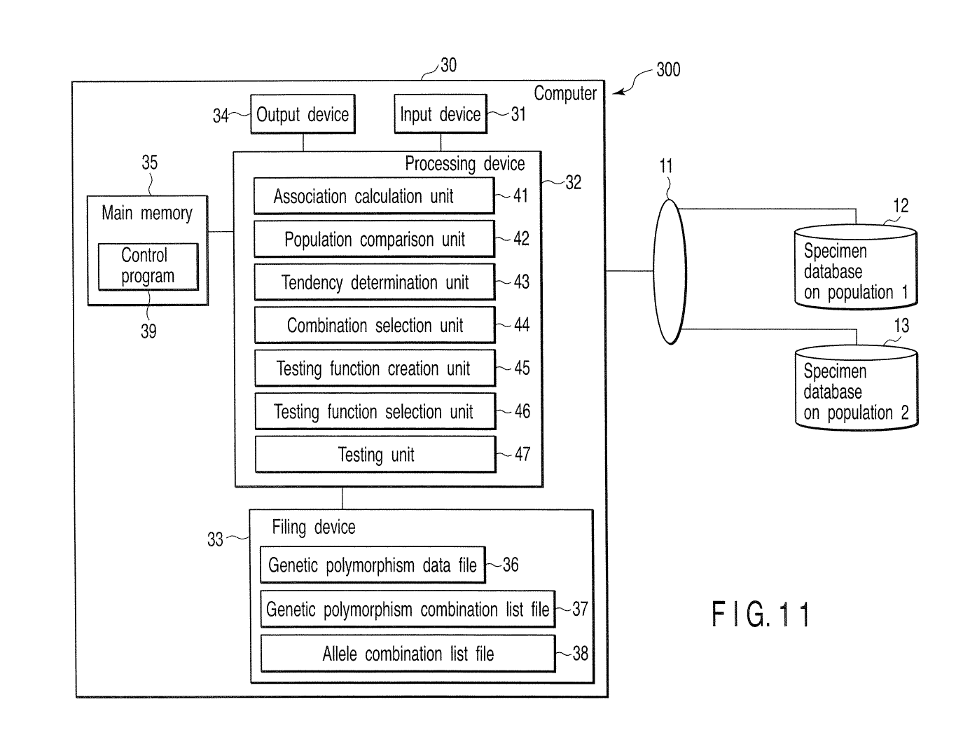

FIG. 11 is a schematic diagram of a genetic diagnosis system 300 for executing a genetic diagnosis program. The genetic diagnosis system 300 includes a computer 30, and a specimen database 12 on population 1 and a specimen database 13 on population 2 which are connected to the computer 30 through a communication network 11.

The computer 30 comprises a processing device 32, and a main memory 35, an input device 31, an output device 34, and a filing device 33 which are connected to the processing device 32. In the present embodiment, the processing device 32 further includes diagnosing unit 47 in addition to association calculation unit 41, population comparison unit 42, tendency determination unit 43, combination selection unit 44, diagnosing function creation unit 45, and diagnosing function selection unit 46. In the present embodiment, the configuration of the computer 30 may be the same as that of the above-descried first and second embodiments, except for the processing device 32.

In the present embodiment, diagnosing functions are created in the same manner as in the second embodiment. Of the created diagnosing functions, an optimum diagnosing function for diagnosis is selected by the diagnosing function selection unit 46. Using the selected diagnosing function, diagnosis is performed on a specimen subject.

First, the gene sequence of a target specimen subject is inputted by use of the input device. In addition, an instruction as to a diagnosis item is inputted. The gene sequence of the target specimen may be only the sequence of a genetic polymorphism used for diagnosing, but it is desirable that the gene sequence is information about all measured genetic polymorphisms.

The diagnosing unit 47 reads a diagnosing function selected by the diagnosing function selection unit 46. Then, the inputted gene sequence of the target specimen is applied to the diagnosing function. Consequently, the expected rate of the specimen for the diagnosis item is calculated. The calculated expected rate is outputted by the output device 34. In this case, the value of the expected rate outputted from the output device 34 is interpreted by a doctor, and a determination is made as to whether or not the treatment is to be provided. Thus, the doctor who makes such a determination requires sufficient expertise to accurately determine the treatment method. For a more convenient method, for example, output may be provided such that when the calculated expected rate for effectiveness is 0.7 or greater, the treatment method is “effective”; when the calculated expected rate for effectiveness is 0.3 or less, the treatment method is “ineffective”; and when the calculated expected rate for effectiveness is between 0.3 and 0.7, the treatment method is “consideration required”. In this case, even if the doctor does not have any particular expertise, he/she can incorporate testing into a standard routine, for example, such that for “effective” the treatment is provided, for “ineffective” the treatment is not provided, and for “consideration required” a determination is made based on the request of a patient.

Although, in the above-described example, the diagnosing function is created by obtaining a linear regression curve, a discriminant may be derived by a discriminant analysis, and the discriminant may be used as a diagnosing function. Discriminant Z=bX+u (where b and u are constants) is a statistical function such that when X obtained by measuring the gene sequence of a specimen is substituted, the treatment method is determined to be effective if Z>0, and the treatment method is determined to be ineffective if Z<0. By creating a system such that, using a discriminant as a diagnosing function, “◯” is outputted if Z>0, and “X” is outputted if Z<0, the doctor can perform testing more easily.

As described above, a target specimen can be diagnosed. Note that the diagnosis item is not limited to the effectiveness of treatment, and may be the tendency to get a disease or the like. By using a diagnosing function derived from an appropriate marker, desired diagnosis can be performed.

Without using a diagnosing function, diagnosis can be easily performed only by whether the specimen is classified into group A or B by a marker. In this case, the diagnosing unit 47 first reads a marker from a third listing created by the tendency determination unit 43. The gene sequence of a target specimen is searched to make a determination as to whether the same genetic polymorphism combination and an allele combination thereof as the marker are exist in the searched sequence. Based on the determination, diagnosis is performed based on the tendency of the used marker relative to a diagnosis item. A diagnosed result is outputted by the output device 34.

Note that, when treatment on a target specimen is completed based on the genetic diagnosis result and a treatment result is found, the new clinical trial data on the specimen can be added to a specimen database. In this case, association listings and other listings can be updated based on the specimen database to which the new specimen data is added, so that the accuracy of a testing system can be further improved.

According to the present invention described above, a marker used for genetic diagnosis can be searched only by computer processing by making the most of already measured genotype data, without performing additional measurement. Accordingly, a considerable amount of time and cost can be saved.

Furthermore, according to the present invention, the following benefits can be obtained.

For example, it is assumed that a clinical trial for genetic diagnosis was performed on population 1, so that specimen data was achieved. In addition, it is assumed that population 2 is a medical institution that actually uses the present genetic diagnosis system based on the specimen data including that of population 1, and provides treatment based on the diagnosis results obtained from the present system. In this case, at an initial stage from the start of genetic diagnosis in the medical institution, the number of specimens in population 2 is smaller than that in population 1. Thus, if populations 1 and 2 are simply combined and treated as a single population and the diagnosing function is updated, the result of population 2 is difficult to be reflected.

If, for some reason, there is a difference in important gene between populations 1 and 2, the specimen data obtained from population 2 that is subjected to actual diagnosis and treatment needs to be more strongly reflected on the specimen data base. However, when populations are simply integrated into a single population, the result of population 2 is difficult to be reflected.

In order to make populations into a single population, all data for each population needs to be stored, increasing the burden for data management. Furthermore, since the total number of specimens in the population increases, a calculation takes more time, and accordingly an update takes more time.

However, according to the present invention which utilizes specimen data on a plurality of populations without integrating all specimen data thereof into single population, even information on a minority population such as population 2 described in the above embodiment can be surely reflected to the diagnosis result. In addition, for population 1, the entire clinical trial data does not need to be stored and only the association listings need to be stored, and therefore, it is also convenient in view of protecting personal information.

More specifically, according to the present invention, an appropriate marker and diagnosing function can be selected without losing the characteristics of each population, and thus, a highly accurate and practical genetic diagnosis system can be provided. In addition, because a specimen database is provided separately for each population, even in the case of adding data on a new specimen to one population, the databases for all the populations do not need to be re-calculated, but only the database of the one population can be re-calculated, and thus, it is convenient and efficient.

Diagnosing functions for genetic diagnosis created according to the present invention can also be stored on a computer-readable storage medium and provided to other medical institution.

In a fourth embodiment, the aforementioned second association listing can be used in searching for a gene which is involved in an onset mechanism or treatment mechanism of a particular disease.

Specifically, a search is made for a signaling pathway through which the genes composing genetic polymorphism combinations listed in the second association listing are linked to each other. As a consequence, it is also possible to clarify a signaling pathway that is not conventionally known. In addition, it is also possible to clarify genes present on the signaling pathway by literature searches or the like.

It is also possible to find a new promising candidate by searching polymorphisms in such genes. This makes it possible to search for a new gene far more efficiently than by conventional methods that are based on researchers' intuition.

The combinations listed in the second association listing can be used regardless of whether the tendencies are the same or different. This is because in gene search, a search for such a signaling pathway that links two genes is possible even if the tendencies of association or signs of correlation coefficients are opposite.

### EXAMPLES

Specific examples of the present invention will be described below, but the present invention is not limited thereto. In the following examples, a marker is selected which is used to diagnose the effectiveness of interferon treatment for hepatitis C patients.

### Example 1

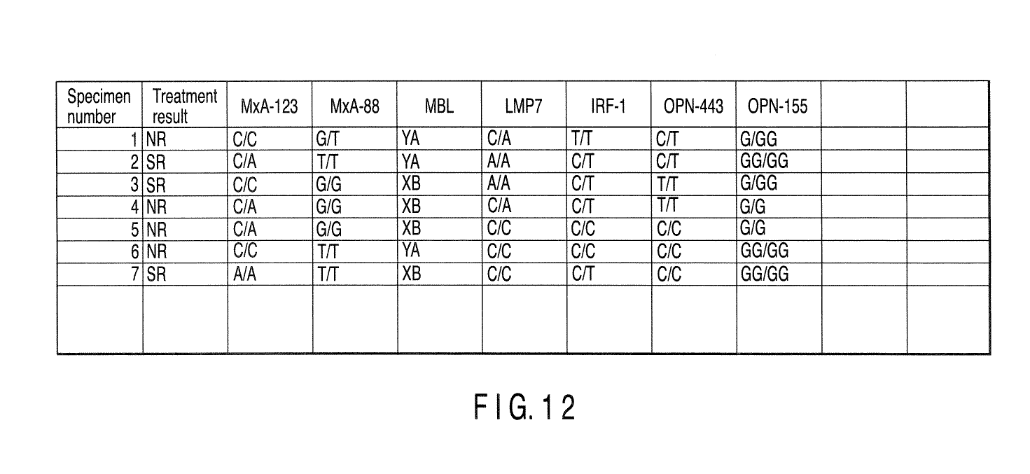

FIG. 12 is a chart showing the contents of a specimen database of hospital T regarding interferon treatment for hepatitis C patients. The results of clinical trials performed in hospital T in the past are stored on the database. The contents of the database of FIG. 12 will be described. An identification number is assigned to a patient subjected to a clinical trial and entered in the “specimen number” column. Note that a specimen number does not correspond with a medical record number in order to protect personal information. A correspondence table between medical record numbers and specimen numbers is managed by a system which is different from the system according to the present invention.

The “treatment result” column shows whether or not the interferon treatment is successful. “SR” indicates that the hepatitis C virus is completely eliminated by the interferon treatment (which is generally called a “sustained response case” or a “response case”). “NR” indicates other case (non-response).

Measured genotypes are entered in the third and subsequent columns. MxA−123 indicates an SNP located at −123 in a promoter region of myxovirus resistance protain A gene, and the base identity thereof takes C or A. There are three types of alleles, C/C, C/A, and A/A. An allele that a specimen has is entered in a corresponding column in the database.

MxA−88 indicates an SNP located at −88 in a promoter region of the MxA gene, and there are three types of alleles, G/G, G/T, and T/T.

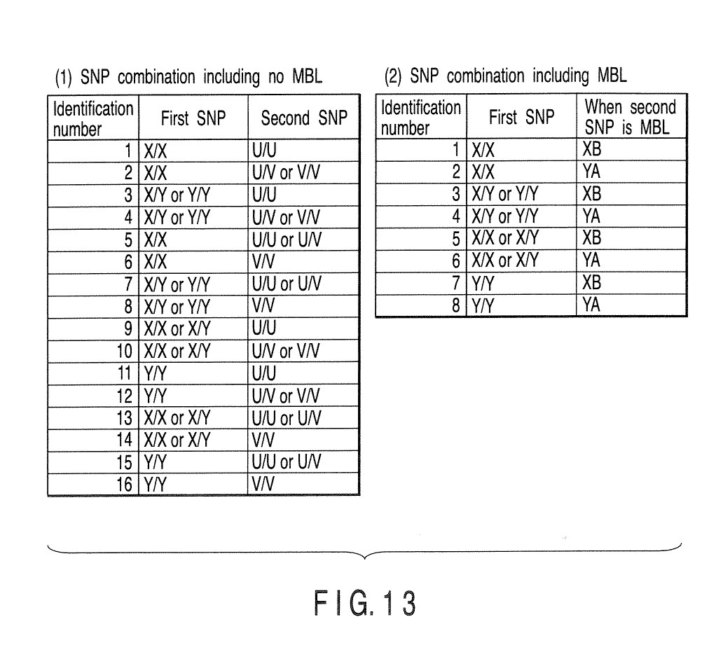

For an allele in an SNP of an MBL gene in the fifth column, there are two types of alleles, YA and XB. In this case, an allele combination list for combinations of an SNP of an MBL and other SNP is as shown in FIG. 13(2).

An allele of an SNP of an LMP7 is C/C, C/A, or A/A. An allele of an SNP of an IRF-1 is C/C, C/T, or T/T. The genotypes of an OPN gene are listed in the eighth and ninth columns. The OPN gene is known to have SNPs at several locations. An SNP located at −443 in a promoter region takes an allele of C/C, C/T, or T/T.

A polymorphism located at −155 in a promoter region of an OPN gene has either a single base G or two bases which are written as G and GG, respectively. The length of a gene is different between the case of G and the case of GG. For an allele, there are three types of alleles, G/G, G/GG, and GG/GG.

Likewise, genotypes are entered in the tenth and subsequent columns but are omitted in FIG. 12. Although FIG. 12 shows clinical trial results in hospital T, similar database is created and stored for hospital S as well.

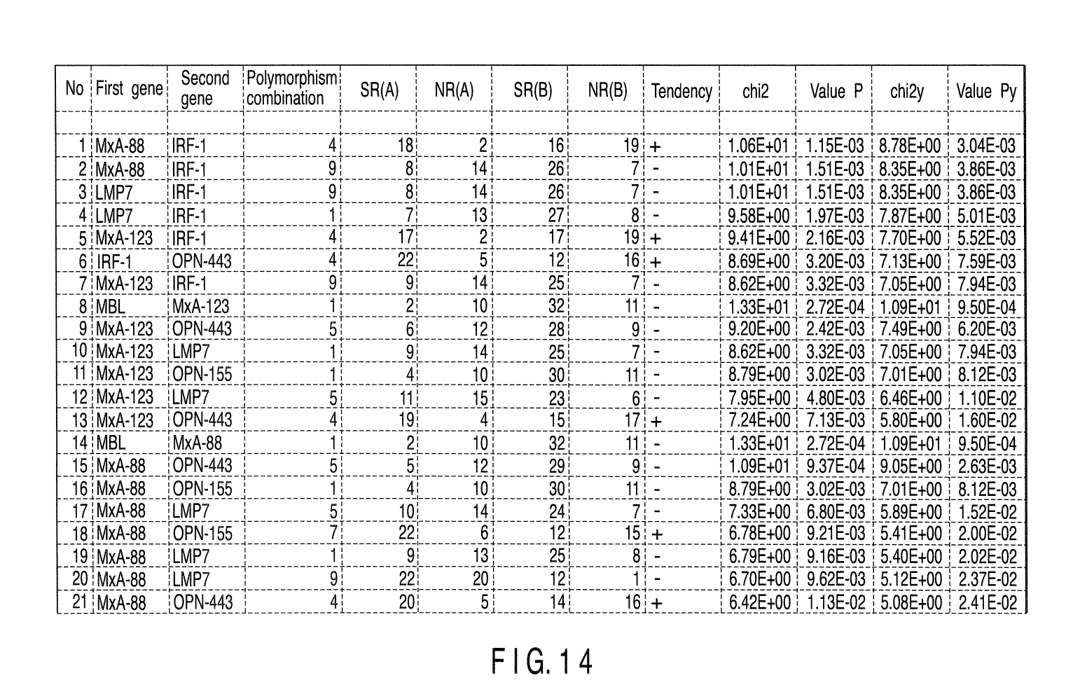

Based on the specimen data database for hospital T and the allele combination list exemplified in FIG. 8, an association with the effectiveness of treatment is calculated by an association calculation unit. As a result, a listing shown in FIG. 14 is created by using combinations having a significant association. FIG. 14 is a chart showing part of the association listing for hospital T.

In the “first gene” and “second gene” columns, the combined two SNPs are identified by gene-names and positions where the respective SNPs are located. In the “polymorphism combination” column, there are listed numbers which indicate how alleles are combined, the each number indicating SNP combination shown in FIG. 13-(2) for the case where one of the genes is MBL, or shown in FIG. 13-(1) for other cases. The polymorphism combination may be identified by numbers with reference to the allele combination list, or an allele representation as described in the allele combination list. “SR(A)” indicates the number of specimens which are classified into group A and which are responsive. Specifically, in the example of No. 1 of FIG. 14, specimens in which the first gene MxA−88 and the second gene IRF-1 satisfy an allele combination of 4 are classified into group A and others are classified into group B. In this case, for specific bases, X=G and Y=T for MxA−88, and U=C and V=T for IRF-1. “NR(A)” indicates the number of specimens which are classified into group A and which are non-responsive. “SR(B)” indicates the number of specimens which are classified into group B and which are responsive. “NR(B)” indicates the number of specimens which are classified into group B and which are non-responsive.

In the “tendency” column, the case where group A has a higher response rate than group B is indicated by “+”, and the opposite case where group A has a lower response rate than group B is indicated by “−”. A chi-square value is listed in the “chi2” column. A value of significant level P obtained by a chi-square test is listed in the “value P” column. A chi-square value with Yates' continuity correction is listed in the next “chi2y” column. A value P obtained from a test with Yates' correction is listed in the “value Py” column. In the present example, it is determined that there is a significant association when value Py<0.05. Likewise, for hospital S whose specimen is grouped into the second population, an association listing for hospital S is created.

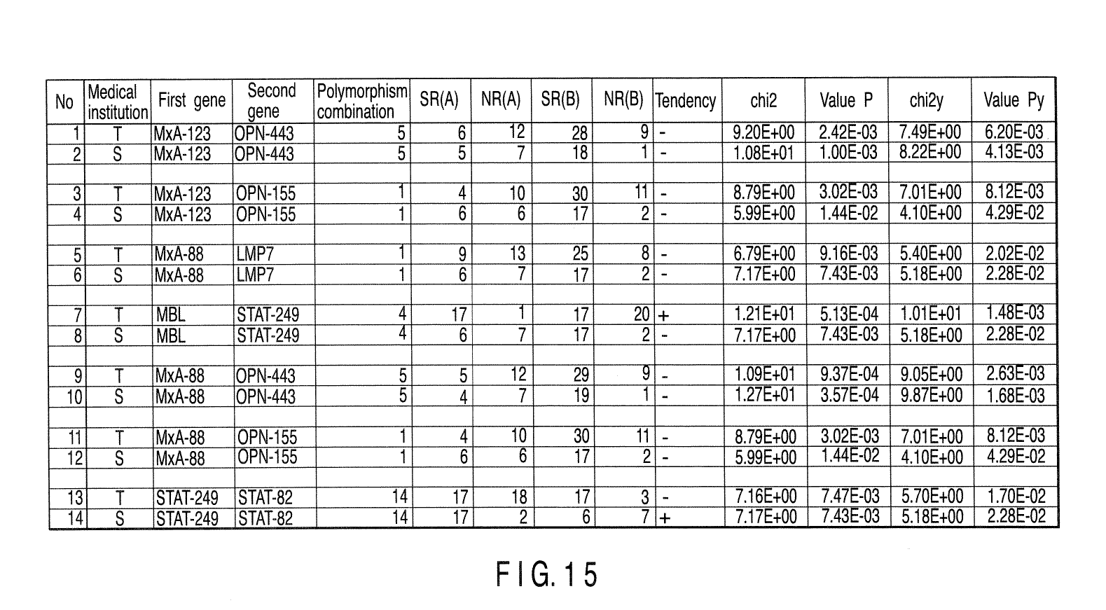

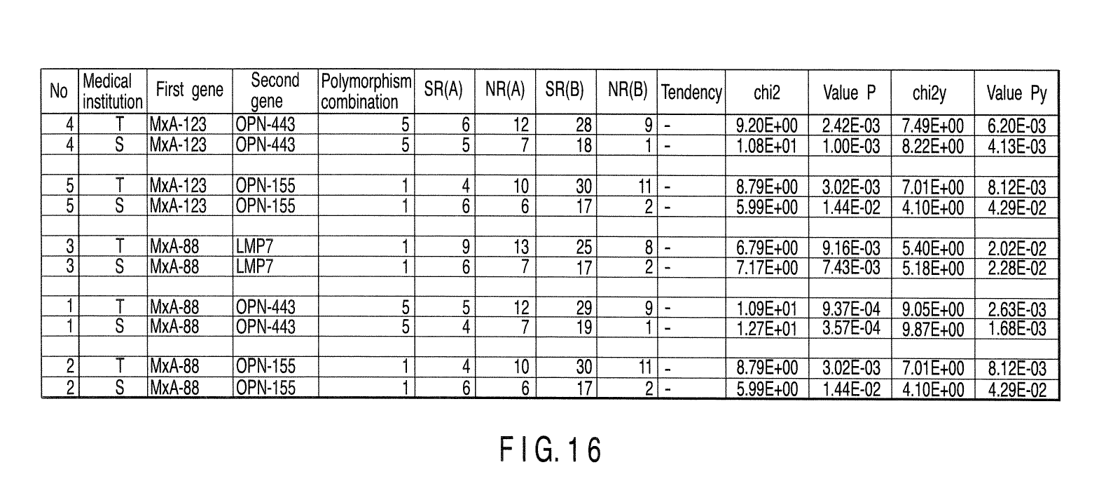

FIG. 15 is a Table showing specific contents of a second association listing. No. 1 and No. 2 indicate the case where MxA−123 and OPN−443 are combined, and further, allele combination of identification number 5 in FIG. 13-(1) is selected. Here, in the present gene combination, X=C, U=C, and V=T. Therefore, when specimens having a combination of MxA−123 which is C/C and OPN−443 which is C/C or C/T are classified into group A and specimens having other SNP combinations are classified into group B, it indicates that there is a significant difference in treatment effect between groups A and B. The tendency is “−” in both hospital T shown in No. 1 and hospital S shown in No. 2. That is, it suggests that interferon is less effective in group A. As such, combinations having the same tendency between hospitals T and S are listed in a third association listing shown in next FIG. 16.

Now, take a look at No. 7 and No. 8 of FIG. 15. Each indicates an SNP combination of an MBL gene and a gene called STAT-249. While the tendency of No. 7 in hospital T is “+”, the tendency of No. 8 in hospital S is opposite to that, i.e., “−”. That is, it shows that both of specimens classified into group A in which MBL and STAT-249 have an allele combination of identification number 4 in FIG. 13(2) and specimens having other allele combination classified into group B have a significant correlation with the effectiveness of interferon in both hospitals T and S. However, in hospital T, interferon is more likely to be effective in group A whereas in hospital S, by contrast, interferon is more likely to be effective in group B. Namely, the tendency is completely opposite for different medical institutions. In this case, such a combination cannot be directly used in genetic diagnosis. Thus, this combination is excluded from being listed in the third association listing shown in FIG. 16.

Note, however, that this result shows the possibility of the presence of a signaling pathway that links MBL and STAT, and may provide valuable knowledge from a medical point of view. In addition, it suggests that the signaling pathway has such characteristics that it sensitively depends on the attribution of the medical institution. Hence, it suggests that there is a possibility that other important genes than 27 genotypes measured in Example 1 may be present in the signaling pathway that links MBL and STAT. The information is important for a new marker search.

In the genetic diagnosis system according to the present invention, the combinations listed in the third association listing shown in FIG. 16 are used as diagnostic markers. In FIG. 16, there are five types of combinations which are indicated by 1 to 5. In the present example, the best combination is selected by a method described below.

Function t1 shown in FIG. 17 is an function obtained by a comparison between the case wherein the genetic polymorphism combination (MxA−88 and OPN−443) of No. 1 of FIG. 16 has a particular allele combination of identification number 5 of FIG. 13-(1) and the case of other combinations. Specifically, a comparison is made between the case wherein the particular combination wherein MxA−88 is G/G and OPN−443 is C/C or C/T, and the case of other combinations.

More specifically, suppose that when the specimen i has such the particular allele combination, xᵢ is numerically represented by −1, and for other cases, xᵢ is numerically represented by +1. In addition, suppose that a specimen i in which the interferon treatment result is responsive is numerically represented such that yᵢ=1 and a specimen in which the interferon treatment result is non-responsive is numerically represented such that yᵢ=0. On these suppositions, the Function t1 shows a linear regression curve in which yᵢ is determined to be associated with xᵢ. A specific linear regression curve for t1 is such that Y=0.261X+0.511. This function is a diagnosing function that provides an expected response rate Y.

For a parameter that evaluates the accuracy of the diagnosing function, the contribution ratio K is defined by the following function. The contribution ratio indicates how much a derived diagnosing function can explain the resultant effectiveness of a treatment method. Summarized in FIG. 17 are the contribution ratios calculated for selecting actual diagnosing marker.

$$K ≡ \frac{{S}_{yy} - {S}_{e}}{{S}_{yy}}$$

$$where$$

$${S}_{e} = {\sum }_{i = 1}^{n} {\{ {y}_{i} - Y ( {x}_{i} ) \}}^{2}$$

is the residual sum of squares, and

$${S}_{yy} = {\sum }_{i = 1}^{n} {( {y}_{i} - \overline{y} )}^{2}$$

is the total sum of squares.

Here, when all data yi is completely explained by diagnosing function Y, K=1 and the contribution ratio is 1. When diagnosing function Y is a linear regression curve, K is equal to the square of a correlation coefficient.

Function t1 of FIG. 17 is a linear regression curve that fits the specimens of hospital T and the contribution ratio for hospital T is 25%. It is examined how much it explains the specimens of hospital S which is a different population by using function t1, and as a result, the contribution ratio is 32%. Function s1 of FIG. 17 is a linear regression curve that fits the specimens of hospital S using a combination of MxA−88 and OPN−443. By use of this regression function as a diagnosing function, it is found that the contribution ratio for the specimen of hospital S is 44% and the contribution ratio for the specimens of hospital T is 15%.

The same calculation is also performed on other gene combinations listed in the third association listing of FIG. 16, and the results are shown in t2 to t5 and s2 to s5 in FIG. 17. In the present example, a gene combination having the maximum average value of calculated four contribution ratios is selected as a new marker.

Specifically, as can be seen in FIG. 17, four contribution ratios for the combination of MxA−88 and OPN−443 are 25%, 32%, 44%, and 15%, respectively. The average value of the contribution ratios is 29% which is the highest in five candidate combinations. Thus, this combination is selected as a marker for genetic diagnosis.

In the present example, only one gene combination is selected as a marker ultimately used. However, it is also possible to create, as a diagnosing function, a linear function in which an appropriate weight is assigned to all the combinations listed in the third association listing. Alternatively, a diagnosing function may be created by using a plurality of arbitrary combinations.

In addition, a single genetic polymorphism may be added to a diagnosing function by a genetic polymorphism combination, and it may be used as a marker. This method is effective for the case, for example, in which there are a plurality of important genes for both hospitals T and S, but the contribution ratios of the genes are low. By adding another marker, the accuracy of the diagnosing function can be increased.

### Example 2

Now, an exemplary case where there are three populations will be described. In the present example as well, a marker is selected which is used to genetically diagnose the effectiveness of interferon treatment for hepatitis C patients. The present example is, however, different from Example 1, because combined therapy with a recently developed new type of interferon and an antiviral drug are used. Since this new technique had not been introduced in Japan at beginning of the trials, analysis was carried out using the results of clinical trials obtained in the U.S.

One of the biggest problems in applying the results of clinical trials obtained in the U.S. to cases in Japan is the difference in race. Moreover, in the U.S., different races are mixed together. Thus, when there is a difference in treatment effect between races, and all data of the different races is integrated into a single population and analyzed, it is highly possible that information on a particular race, especially, a race having a small number of data, may be buried and lost.

The results of clinical trials used this time are obtained from 150 specimens in total, which include three types of races, White, Black, and Native Americans. The breakdown is as follows: 60 White specimens, 70 Black specimens, and 20 Native American specimens. Since Japanese are considered to be closest to Native Americans, it is considered that prim importance should be placed on information from Native Americans.

However, the number of Native American specimens is smaller than that of White specimens or Black specimens. For this reason, when all these 150 specimens are analyzed as a single population, a tendency that is strongly found only in Native Americans may be hidden. However, by using a technique of the present invention, the results of clinical trials obtained in the U.S. can be effectively used for predicting the effectiveness of a new treatment method when applied to Japanese.

Specifically, specimen data is classified into races, thereby obtaining three populations of White, Black, and Native Americans. In a White specimen database, there are stored the results of SNPs identification at eight locations which are likely to be associated with the new treatment method and the results of treatment showing whether the new treatment method is effective or not for each specimen for the 60 White specimens.

Likewise, a Black specimen database has stored thereon identities of SNPs at eight locations and the results of treatment for the 70 Black specimens. In a Native American specimen database, SNP data and the results of treatment for the 20 Native American specimens are stored.

Note that the SNPs at eight locations that are identified this time are referred to as “a to h”. Of the SNPs at eight locations, any two SNPs are combined and a genetic polymorphism combination list in which (8×7)/2=28 types of SNP combinations are listed is created. Furthermore, a list of allele combinations that correspond to genetic polymorphism combinations in the genetic polymorphism combination list is created.

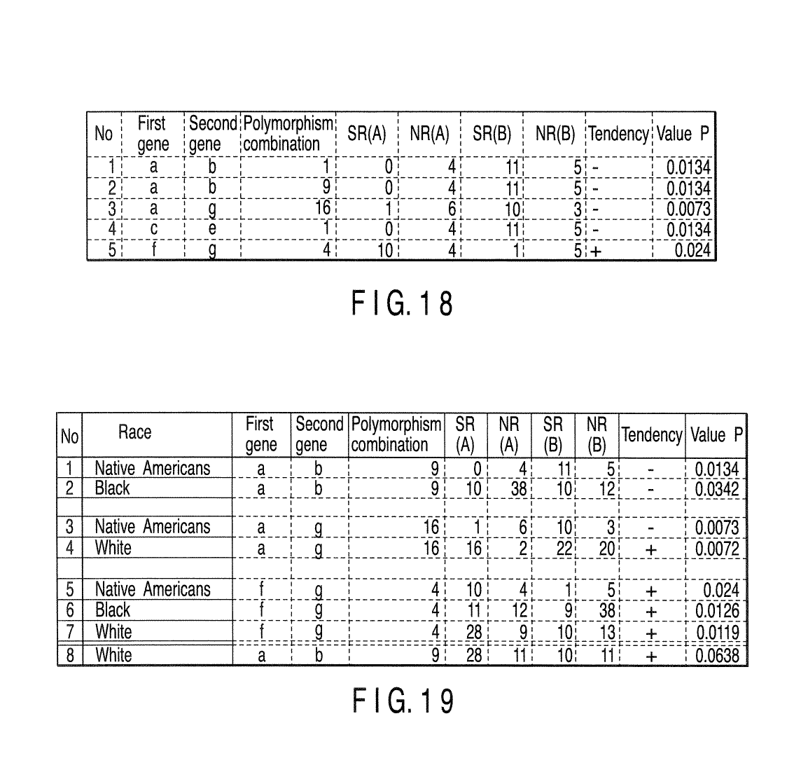

First, the association between genotype combinations and the effectiveness of treatment is calculated by use of data in the Native American specimen database. In this analysis, a chi-square test is performed, and when the value P is 0.05 or less, it is determined that there is a significant association. The results are shown in FIG. 18. An association listing of FIG. 18 is an “association listing for Native Americans”.

“No.” indicates a serial number. The names of genes in which each of the two combined genetic polymorphisms are located are listed in the “first gene” and “second gene” columns. A number indicating the combination of alleles is listed in the “polymorphism combination” column. For the specific contents of the “polymorphism combination”, reference is made to the allele combination list. In the present example, the list of FIG. 13-(1) can be used as the reference. As in Example 1, specimens having a specified polymorphism combination are classified into group A, and other specimens are classified into group B. SR(A), NR(A), SR(B), and NR(B) respectively indicate, as in Example 1, the number of specimens which are classified into group A and which are responsive, the number of specimens which are classified into group A and which are non-responsive, the number of specimens which are classified into group B and which are responsive, and the number of specimens which are classified into group B and which are non-responsive, respectively.

In the “tendency” column, the case where group A has a higher response rate than group B is defined by “+”, and the case where group B has a higher response rate than group A is defined by “−”.

The value P means value of significance level P in the chi-squared test of independence. As described above, in the present example, the case where the value P is 0.05 or less is regarded to have a significant association and thus is listed in the association listing. However, the present invention is not limited thereto, and as in Example 1, value P obtained from a test with Yates' correction may be used for determination, or other references may be used.

By the same steps, an association listing for White is created by using the White specimen data database. Likewise, an association listing for Black is created by using the Black specimen data database. Then, SNP combinations found to have a significant association between all different races are extracted by the population comparison unit, and a second association listing is created. Specific contents of the second association listing according to the present example are shown in FIG. 19.

Since there are two populations in previous Example 1, SNP combinations that are determined to have a significant association between the two populations are listed in the second association listing. In the present Example 2, however, there are three populations. Thus, only SNP combinations that are determined to have a significant association between all of the three populations can be listed in the second association listing. In the present example, however, the following strategy is used for the selection of SNPs to be listed in the second association listing for the following reason.

As described above, the purpose of this analysis is to use the results thereof in the future for the treatment of Japanese patients with hepatitis C. It is expected that the treatment effect greatly differs between races. For example, in the present example, White have 38 response cases and 22 non-response cases and the response rate is 63% while Black have 20 response cases and 50 non-response cases and the response rate is 29%. It can be seen that White and Black are populations between which the response rate is significantly different (P=0.00007).

Native Americans have 11 response cases and 9 non-response cases, and the response rate is 55%. Thus, there is a significant difference (P=0.028) in response rate between Native Americans and Black. Accordingly, it is naturally expected that it is highly possible that the effectiveness of the new treatment method varies between races.

In general, Japanese are considered to be closest to Native Americans in terms of the race. Thus, great importance is placed on the SNP combinations that were found to have a significant association in Native Americans. Hence, by use of the population comparison unit, a program is set to select common SNP combinations which have been found to have a significant association in both Native Americans and at least one other population.

Therefore, important combinations common in both Native Americans and White, important combinations common in both Native Americans and Black, or common combinations having a significant association in all of the three populations of Native Americans, Black, and White, are listed in the second association listing shown in FIG. 19.

In FIG. 19, No. 1 and No. 2 each show a combination of genetic polymorphisms (SNPs) a and b. This allele combination corresponds to a combination of identification number 9 in FIG. 13. A significant association between the effectiveness of the new treatment method and the SNP combination is found in the population of Native Americans indicated by No. 1 and the population of Black indicated by No. 2. In this association, group B has a higher response rate than group A in both Native Americans and Black. Since the tendencies of the association in both populations correspond with each other, No. 1 and No. 2 are also listed in a third association listing.

For this SNP combination, a significant association with effectiveness of the new treatment method is not found in the population of White. For reference, it is shown in No. 8. In the population of White, the value P is 0.0638 which is greater than 0.05, and thus it is determined that there is no association.

No. 3 and No. 4 each show a combination of genes a and g in the populations of Native Americans and White, and have an allele combination of identification number 16 in FIG. 13(1). This combination shows that there is a significant association with the effectiveness of the new treatment method. However, Native Americans have a tendency of “−” whereas White, by contrast, has a tendency of “+”. Thus, this combination is not listed in the third association listing.

Nos. 5 to 7 each show a combination found to have a significant association with the effectiveness of the new treatment method in all of the three populations of Native Americans, White, and Black. Since all of the three populations have a tendency of “+”, these are also listed in the third association listing.

In the genetic diagnosis system of the present invention, the genetic polymorphism combinations listed in the third association listing are used as markers. Therefore, two types of combinations are used as markers, i.e., one is a combination of genes a and b having an allele combination of identification number 9, and the other one is a combination of genes f and g having an allele combination of identification number 4. Hereinafter, the former one is referred to as the “combination U”, and the latter one is referred to as the “combination V”.

Then, a selection is made so as to determine one combination which can be most suitably used by use of the candidate selection unit. However, since, in the present example, there are only two combinations found as candidates, these two combinations are used as variables to create a diagnosing function by the diagnosing function creation unit.

First, when gene combination U of the i-th specimen is classified into group A, numerical representation uᵢ=1 is employed. When classified into group B, numerical representation uᵢ=−1 is employed. Likewise, when the i-th specimen gene combination V is classified into group A, vᵢ is numerically represented by 1. When classified into group B, vᵢ is numerically represented by −1. Furthermore, the result of treatment of each specimen i is represented by yᵢ, and the case where the new treatment method is effective is represented such that yᵢ=1, and the case where the new treatment method is ineffective is represented such that yᵢ=0.

By the above-described numerical representation, a multiple linear regression curve Y=au+bv in which yᵢ is predicted from uᵢ and vᵢ is determined and used as diagnosing function Y. Diagnosing function Y thus determined provides an expected response rate for a given case of certain U and V.

In the actual diagnosing system, gene combinations U and V of unknown specimens are measured and substituted in diagnosing function Y=au+bv. The system is set so that a genetic diagnosis result is outputted as a result of the substitution as follows: the new treatment method is ineffective when Y is less than 0.3, the new treatment method is uncertain when Y is 0.3 or greater and less than 0.7, and the new treatment method is effective when Y is 0.7 or greater.

This system is actually used in a medical institution in Japan, and as a result, the contribution ratio of the diagnosing function is 60%, providing a very good result. Thus, it has been established that such a high-accuracy diagnosing function can be obtained according to the method of the present invention. The reason for which a high-accuracy diagnosing function can be obtained according to the present invention will now be described as follows.

In general, upon performing statistical processing, a higher accuracy statistical processing can be done with a greater number of data. However, in a new field such as genetic diagnosis, the human genome has not yet been completely elucidated. The disease incidence or the effectiveness of a treatment method is not determined only by genes and also depends on various environmental factors such as diet, amount of exercise, past history, and complications.

Hence, it is not always proper to consider whole specimens as the same population. Depending on a matter to be diagnosed, effective information may be obtained by considering an environmental factor that is likely to affect, and dividing specimens into plural populations.

In the present example, considering the fact that clinical trial data obtained in the U.S. includes data on various races, specimens are divided into three populations of White, Black, and Native American. Here, take a note of No. 1, No. 2 and No. 8 in the second association listing shown in FIG. 19. No. 1 and No. 2 each show the case of having a gene combination of a and b, and an allele combination of 9. In this case, a significant association with the effectiveness of the treatment method is found in both the populations of Native Americans and Black. However, in White of No. 8, P=0.0638, and thus a significant association is not found.

In the present example, the specimens are divided by race into three populations. Furthermore, since Japanese are considered to be close to Native Americans, great importance is placed on Native Americans and there are used combinations found in both the population of Native Americans and other one population.

On the other hand, assuming that analysis is carried out based on a principle that a greater number of specimens are better, data on 150 specimens will be all included in a single population. In that case, SR(A)=38, NR(A)=53, SR(B)=31, and NR(B)=28 in the combination shown in No. 1 and No. 2. Group A has a response rate of 42% while group B has a response rate of 53%, and thus there is not much difference between the two groups. When a chi-square test is actually performed, the result is P=0.195, reaching a conclusion that there is no association between this SNP combination and the effectiveness of the new treatment method. That is, the conclusion means that the SNP combination of U cannot be found by prior arts.

Furthermore, assuming that the combination U of No. 1 and No. 2 is not present, a diagnosing function is created on the supposition that only the combination V of No. 5, No. 6, and No. 7 is a candidate marker. Using this resultant diagnosing function, the treatment data obtained by a medical institution in Japan are analyzed. As a result, the contribution ratio thereof is a little less than 30%.

As described above, the use of the present invention makes it possible to select a candidate marker that is not recognized by conventional methods, and to provide a high-accuracy diagnosing function, diagnosing method, and system.

### Example 3

An example according to the third embodiment of the present invention will be described. Specifically, a method will be described in which after selecting candidate markers associated with the effectiveness of interferon in Example 1 (the third association listing), the markers are further narrowed down based on biological knowledge.

Here, for a method of narrowing down candidate markers associated with the effectiveness of interferon based on biological knowledge, a system biological technique using literature information is used.

It is known that when interferon acts on cultured cells, a series of reactions, called an “interferon signaling pathway”, took place in the cells. It is considered that during treatment using interferon, this signaling pathway is mainly acting.

From this fact, it is presumed that a gene associated with the effectiveness of interferon treatment or a protein that is a product of the gene is involved in an intracellular system including the interferon signaling pathway (referred to hereinafter as the “interferon signaling system”).

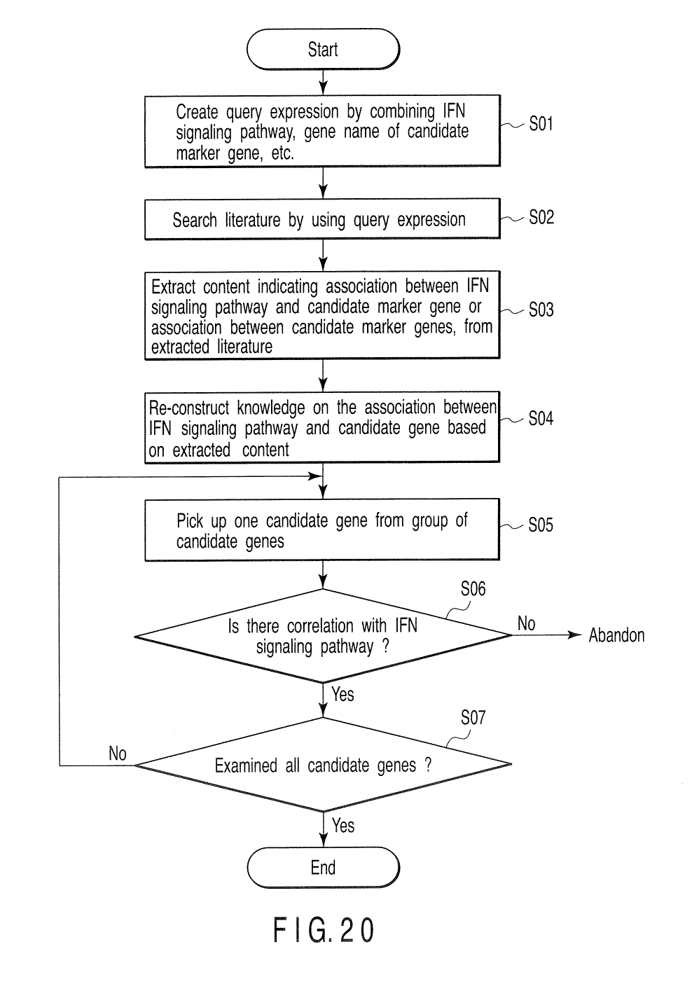

Thus, in the present example, the candidate markers listed in the third association listing are further narrowed down by using the presence/absence of an association with the interferon signaling system as a pilot. A flowchart of this process is shown in FIG. 20.

First, biological knowledge about the interferon signaling system is collected (S01 to S04). In the present example, a keyword search is performed on PubMed (http://www.ncbi.nlm.nih.gov/) which is the largest medical and biological literature database.

In step S01, a query expression is created by using, as a key words, a gene name associated with the interferon signaling system, a protein name, gene names of candidate markers having been further narrowed down by a statistical technique, and the like which are appropriately combined.

In step S02, the query expression is sent to PubMed, and relevant literatures are collected. In step S03, contents regarding the association between the interferon signaling system and the candidate markers in the third association listing are extracted from the collected literatures.

The extraction method includes, for example, a method in which a specialist performs extraction and an extraction method in which a computer performs extraction using natural language processing technology. With the former method, the accuracy of extracted information can be expected. With the latter method, a vast amount of literature can be processed in a short time. The extracted contents are stored in a format that can be processed by the computer.

In steps S04 to S07, the correlation between all of the markers and the interferon signaling system is organized based on the informations regarding the association between the interferon signaling system and the candidate marker genes which are extracted in step S03. A determination is made as to whether each candidate marker gene has an association with the interferon signaling system.

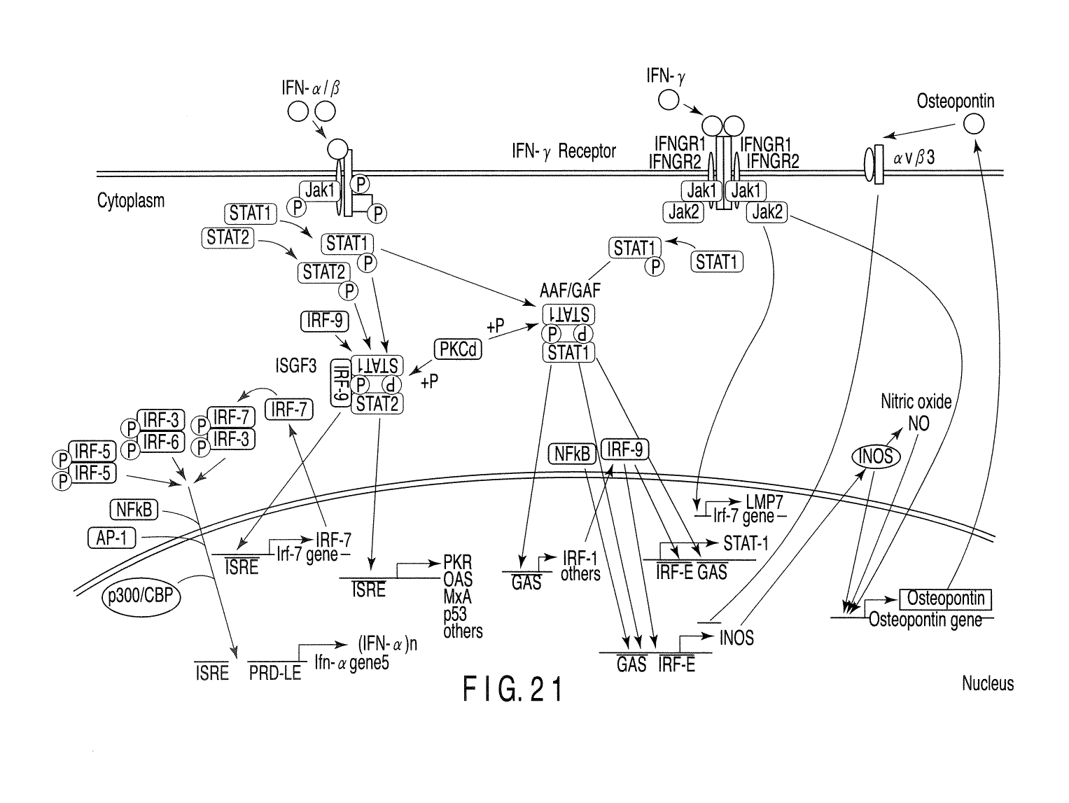

A schematic diagram showing the correlation between the interferon signaling system and the markers which is obtained in the present example is provided in FIG. 21.

From FIG. 21, it has been found that all of the candidate markers, except for MBL, have a correlation with the interferon signaling system at the time of carrying out the present example.

Particularly, OPN is found to have the effectiveness of interferon, but has not so far been clear about an association with the interferon signaling system previously. However, it has been shown by the present example that OPN has an association with the interferon signaling system. This shows the effectiveness of the present method.

On the other hand, in the present analysis, MBL is not found to have a correlation with the interferon signaling system, and therefore, MBL is excluded from being a candidate. However, this may be due to the fact that the transcriptional regulation of MBL gene was not yet elucidated at the time of carrying out the present example. In the future, when biological knowledge about the transcriptional regulation of MBEL is accumulated, MBL may be found to have a correlation with the interferon signaling system by carrying out analysis again by use of the method of the present example.

As such, use of the method according to the present example makes it possible to further narrow down the candidate markers having been selected by a statistical technique to markers that are also supported by biological knowledge. Accordingly, interferon effective genes can be screened with higher accuracy.

### Example 4

An example according to the fourth embodiment of the present invention will be described. Specifically, a method will be described in which after selecting candidate markers associated with the effectiveness of interferon in Example 1 (the third association listing), the markers are further narrowed down based on biological knowledge.

Here, genetic polymorphisms associated with the effectiveness of interferon are narrowed down by making full use of biological knowledge about the transcriptional regulation.

When genetic polymorphisms are involved in the effectiveness of interferon, two types of action mechanisms are principally considered. One mechanism is that the polymorphism changes the amount of gene expression by acting on the gene transcriptional regulation. In the other mechanism, the polymorphism changes the amino-acid sequence of a protein coded by the gene, thereby resulting in a change of the function of the protein. Recently, it has been noted that the action of a genetic polymorphism such as the former one may be involved in individual difference in the effectiveness or side effect of a drug. The present example also proposes a system particularly specialized for the former case.

In the transcriptional regulation, control through a protein called a “transcription factor” is well known. The transcription factor is a protein that recognizes a particular DNA sequence in a genome and is bound thereto. The transcription factor is attached to a region associated with transcriptional regulation of each gene, thereby promoting or suppressing the transcription of a particular gene.

Different transcriptional regulation may be resulted depending on a base (called “allele”) that the polymorphism takes, when a genetic polymorphism is present in a region to which a transcription factor is bound. Such a result may be caused in the case that (1) the binding ability of the transcription factor is changed; or (2) the transcription factor to be bound is changed to another one, both depending on the allele. In this case, it can be construed that the genetic polymorphism acts on the transcriptional regulation.

If such a polymorphism that possibly acts on the transcriptional regulation is included in the genetic polymorphisms having been statistically narrowed down, i.e., the genetic polymorphisms of candidate markers in the third association listing, it is considered that such a polymorphism has a high probability of becoming a main factor associated with the effectiveness of interferon.

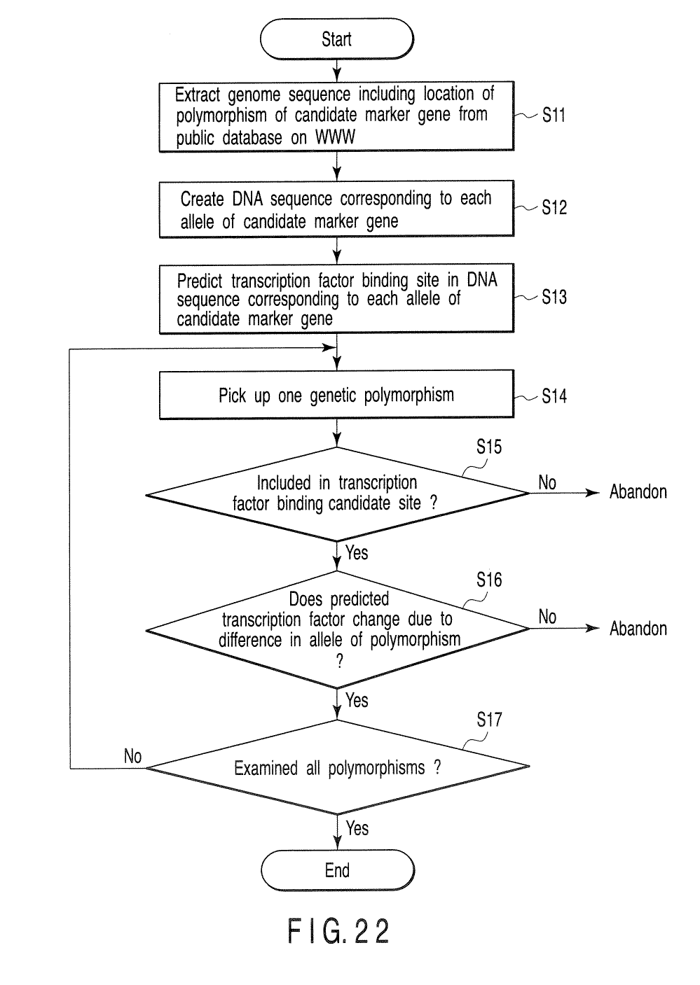

The present example takes note of this point, and provides a method of picking up genetic polymorphisms associated with the effectiveness of interferon. Specifically, in case of considering the candidate markers in the third listing, a genetic polymorphism among the candidate markers is concluded to have a high probability of being a main factor associated with the effectiveness of interferon when the following two conditions are fulfilled. The conditions are (1) a genetic polymorphism is present in a candidate region to which a transcription factor is bound; and (2) the transcription factor predicted to be bound to the candidate region is changed depending on the allele of the polymorphism. A flowchart of such a process according to the present example is shown in FIG. 22.

In the present example, first, the genomic sequences of candidate marker genes having been statistically narrowed down as described above are obtained from a public database (step S11). In this example, Ensembl (http://www.ensembl.org/) is used for a public genome database.

Specifically, a search is performed on Ensembl using the gene names of the candidate marker genes as keywords, thereby obtaining data on the genes stored in Ensembl. The DNA sequences of regions including both each gene and a polymorphism in the vicinity of the gene are obtained from the data.

Based on the obtained DNA sequences of the genes, DNA sequences that correspond to alleles of the genes are created (step S12).

In the subsequent step S13, a prediction is made on candidate for transcription factor binding site, which are included in the DNA sequences. For the prediction of the candidate for transcription factor binding site included in the DNA sequences, there are several methods already developed. In this example, ConSite (http://mordor.cgb.ki.se/cgi-bin/CONSITE/consite/) is used. This Web site provides services on the Web to make a prediction of candidates for transcription factor binding site. The DNA sequences created in step S12 are sent to the site of ConSite, a prediction is made on candidate sites for transcription factor binding which are included in the DNA sequences, and results of the prediction are stored locally.

Then, each of the predicted candidate sites for transcription factor binding is examined to see whether or not the polymorphism of each gene is included in the sites (step S15). If the polymorphism is included in a candidate site, it is further examined to see whether a transcription factor predicted to be bound on the site changes depending on each allele of the polymorphism (step S16). If the predicted transcription factor changes, the polymorphism of the gene is picked up as a genetic polymorphism associated with the effectiveness of interferon.

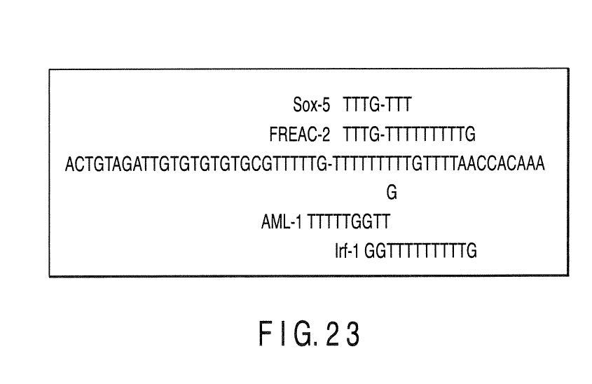

FIG. 23 shows OPN−155 which is one of the polymorphisms picked up in this example. In this polymorphism, an allele in which guanine is missing and an allele in which guanine is contained are present. Due to this difference between the alleles, a transcription factor predicted to be bound is different in some cases. FIG. 23 shows the names of four types of transcription factors predicted to be bound only to one of alleles and sequences recognized by the transcription factors. It is shown by wet experiment that one of the transcription factors, AML-1, is actually involved in the transcriptional regulation of OPN, showing the effectiveness of the method of the present example.

What is claimed is:

1. A marker selection program which causes a computer to work as functional parts for selecting a marker for use in genetic diagnosis, the functional parts comprising:
a genetic polymorphism data storage configured to store in advance known genetic polymorphisms;
a genetic polymorphism combination list storage configured to store in advance a list of genetic polymorphism combinations each composed of at least two genetic polymorphisms included in data on the genetic polymorphisms;
an allele combination list storage configured to store in advance a list of an allele combination regarding the genetic polymorphism combinations listed in the list of genetic polymorphism combination;
a specimen data storage configured to store, for each of at least two populations to each of which a plurality of specimens belong, a genotype of each specimen assigned from the known genetic polymorphisms and a tendency thereof with regard to a diagnosis item;
an association calculation unit configured to store an allele combination list regarding each genetic polymorphism combination listed in the list of genetic polymorphism combination list and determining whether or not allele combinations listed in the list have a correlation with the diagnosis item based on data stored in a specimen database;
an association listing storage configured to store, in an association listing for each population, the genetic polymorphism combinations and the allele combinations thereof determined to have a correlation by the association calculation unit;
a population comparison unit configured to compare between the association listings for the populations and storing, in a second association listing, the genetic polymorphism combinations and the allele combinations thereof, which are present in all of at least two association listings;
a tendency determination unit configured to select, from the second association listing, the genetic polymorphism combinations and the allele combinations thereof having a same tendency against the diagnosis item in at least two populations, and listing the selected combinations in a third association listing as candidate markers; and
an output unit configured to output the candidate markers obtained by the tendency determination unit.

2. The marker selection program according to claim 1, wherein the association calculation unit performs:
reading the allele combination list for each genetic polymorphism combination in the genetic polymorphism combination list,
classifying specimens based on the specimen database, specimens having each allele combination in the list being classified into group A and other specimens being classified into group B,
classifying specimens both in the groups A and B into an effective group and an ineffective group according to the tendency of the diagnosis item,
testing to determine whether or not there is a significant difference in ratio between the effective and ineffective groups in the case for groups A and for B, and
making an judgment that the genetic polymorphisms and alleles determined to have a significant difference by the test have an association with the diagnosis item.

3. The marker selection program according to claim 1, wherein said functional parts further comprise, after the tendency determination unit:
a candidate selection unit configured to select an optimum candidate marker for genetic diagnosis from the candidate markers listed in the third association listing.

4. The marker selection program according to claim 3, wherein the candidate selection unit is a unit configured to average correlation coefficients of the populations and to select a genetic polymorphism combination and an allele combination thereof having a maximum average value of the correlation coefficient.

5. A diagnosing function creation program which causes a computer to work as functional parts for creating a genetic diagnosing function, the functional parts comprising:
a diagnosing function creation unit configured to create, for each population and for the candidate markers in the third association listing of claim 1 or 2, a diagnosing function Y=aX+t (where a and t are constants) by setting X=xᵢ=−1 for specimen i belonging to the group A and setting X=xⱼ=+1 for specimen j belonging to the group B, or setting X=xᵢ=+1 for the specimen i belonging to the group A and setting X=xⱼ=−1 for the specimen j belonging to the group B, or setting X=xᵢ=α for the specimen i belonging to the group A and setting X=xⱼ=β for the specimen j belonging to the group B (where α and β are any different numbers), and setting yᵢ=1 or yᵢ=0 for each specimen i based on effectiveness of treatment and/or a tendency to get a disease; and
an output unit configured to output the created diagnosing functions.

6. The diagnosing function creation program according to claim 5, wherein said functional parts further comprise, after functioning as the diagnosing function creation unit:
a calculation unit configured to calculate a contribution ratio K of the diagnosing function to each population;
a selection unit configured to select a diagnosing function of a candidate marker having a maximum average value of the contribution ratio K, thereby to select an optimum diagnosing function; and
an output unit configured to output the selected diagnosing function, wherein
the contribution ratio K is a parameter which evaluates accuracy of the diagnosing function and is expressed by:
$$K ≡ \frac{{S}_{yy} - {S}_{e}}{{S}_{yy}}$$
$$where$$
$${S}_{e} = {\sum }_{i = 1}^{n} {\{ {y}_{i} - Y ( {x}_{i} ) \}}^{2}$$
is a residual sum of squares, and
$${S}_{yy} = {\sum }_{i = 1}^{n} {( {y}_{i} - \overline{y} )}^{2}$$
is a total sum of squares.

7. A genetic diagnosis system by using a marker selected in any one of claims 1 to 4 for performing genetic diagnosis on target specimens, the system comprising:
a reading unit configured to read the marker selected in any one of claims 1 to 4;
an input unit configured to input respective gene sequences of the target specimens which are measured in advance;
a determination unit configured to determine whether or not a genetic polymorphism combination and an allele combination thereof, which are the same as the selected marker, are present in the specimens;
a diagnosing unit configured to perform diagnosis on the specimens based on the determination; and
an output unit configured to output results of the diagnosis.

8. A genetic diagnosis system for performing genetic diagnosis on target specimens by using diagnosing functions created in claim 5, the system comprising:
a reading unit configured to read the diagnosing functions created as described above;
an input unit configured to input respective gene sequences of the target specimens which are measured in advance;
an applying unit configured to apply data on the specimens to the diagnosing functions and obtaining an expected rate; and
an output unit configured to output the obtained expected rate.

9. A marker selection apparatus for selecting a marker for use in genetic diagnosis, the apparatus comprising:
a genetic polymorphism data storage configured to store in advance known genetic polymorphisms;
a genetic polymorphism combination list storage configured to store in advance a list of genetic polymorphism combinations each composed of at least two genetic polymorphisms included in data on the genetic polymorphisms;
an allele combination list storage configured to store in advance a list of an allele combination regarding the genetic polymorphism combinations listed in the list of genetic polymorphism combination;
a specimen data storage configured to store, for each of at least two populations to each of which a plurality of specimens belong, a genotype of each specimen assigned from the known genetic polymorphisms and a tendency thereof with regard to a diagnosis item;
an association calculation unit configured to store an allele combination list regarding each genetic polymorphism combination listed in the list of genetic polymorphism combination list and determining whether or not allele combinations listed in the list have a correlation with the diagnosis item based on data stored in a specimen database;
an association listing storage configured to store, in an association listing for each population, the genetic polymorphism combinations and the allele combinations thereof determined to have a correlation by the association calculation unit;
a population comparison unit configured to compare between the association listings for the populations and storing, in a second association listing, the genetic polymorphism combinations and the allele combinations thereof, which are present in all of at least two association listings;
a tendency determination unit configured to select, from the second association listing, the genetic polymorphism combinations and the allele combinations thereof having a same tendency against the diagnosis item in at least two populations, and listing the selected combinations in a third association listing as candidate markers; and
an output unit configured to output the candidate markers obtained by the tendency determination unit.

10. The marker selection apparatus according to claim 9, wherein the association calculation unit performs:
reading the allele combination list for each genetic polymorphism combination in the genetic polymorphism combination list,
classifying specimens based on the specimen database, specimens having each allele combination in the list being classified into group A and other specimens being classified into group B,
classifying specimens both in the groups A and B into an effective group and an ineffective group according to the tendency of the diagnosis item,
testing to determine whether or not there is a significant difference in ratio between the effective and ineffective groups in the groups A and B, and
making an judgment that the genetic polymorphisms and alleles determined to have a significant difference by the test have an association with the diagnosis item.

11. The marker selection device according to claim 9, further comprising a candidate selection unit configured to select an optimum candidate marker for genetic diagnosis from the candidate markers listed in the third association listing.

12. The marker selection device according to claim 11, wherein the candidate selection unit is a unit configured to average correlation coefficients of the populations and to select a genetic polymorphism combination and an allele combination thereof having a maximum average value of the correlation coefficient.

13. An apparatus for creating a genetic diagnosing function, comprising:
a diagnosing function creation unit configured to create, for each population and for the candidate markers in the third association listing of claim 1 or 2, a diagnosing function Y=aX+t (where a and t are constants) by setting X=−1 for specimens belonging to the group A and setting X=+1 for specimens belonging to the group B, or setting X=+1 for the specimens belonging to the group A and setting X=−1 for the specimens belonging to the group B, or setting X=α for the specimens belonging to the group A and setting X=β for the specimens belonging to the group B (where α and β are any different numbers), and setting y=1 or y=0 for each specimen based on effectiveness of treatment and/or a tendency to get a disease; and
an output unit configured to output the created diagnosing functions.

14. The genetic diagnosing function creation apparatus according to claim 13, further comprising:
a calculation unit configured to calculate a contribution ratio K of the diagnosing function to each population;
a selection unit configured to select a diagnosing function of a candidate marker having a maximum average value of the contribution ratio K, thereby to select an optimum diagnosing function; and
an output unit configured to output the selected diagnosing function, wherein
the contribution ratio K is a parameter which evaluates accuracy of the diagnosing function and is expressed by:
$$K ≡ \frac{{S}_{yy} - {S}_{e}}{{S}_{yy}}$$
$$where$$
$${S}_{e} = {\sum }_{i = 1}^{n} {\{ {y}_{i} - Y ( {x}_{i} ) \}}^{2}$$
is a residual sum of squares, and
$${S}_{yy} = {\sum }_{i = 1}^{n} {( {y}_{i} - \overline{y} )}^{2}$$
is a total sum of squares.

15. A computer-readable storage medium having stored thereon diagnosing functions for genetic diagnosis which are created in claim 5.
